SEQUENCE LISTING (1 sequence)

SEQ ID NO: 1 - protein, 11 aa, Artificial Sequence
  Synthetic
QQKFQFQFEQQ

(19) United States
(12) Patent Application Publication
(10) Pub. No.: US 2024/0131079 A1
(43) Pub. Date: Apr. 25, 2024

(54) EXTRACELLULAR VESICLES DERIVED FROM INDUCED PLURIPOTENT AND EMBRYONIC STEM CELLS, AND METHODS OF USE FOR IMMUNE MODULATION

(71) Applicant: The Regents of the University of California, Oakland, CA (US)

(72) Inventors: Jana ZARUBOVA, Los Angeles, CA (US); Mohammad Mahdi HASANI-SADRABADI, Los Angeles, CA (US); Song Li, Los Angeles, CA (US)

(73) Assignee: The Regents of the University of California, Oakland, CA (US)

(21) Appl. No.: 18/278,771

(22) PCT Filed: Mar. 4, 2022
(86) PCT No.: PCT/US22/18997

§ 371 Date: Aug. 23, 2023

Related U.S. Application Data
(60) Provisional application No. 63/157,087, filed on Mar. 5, 2021.

(51) Int. Cl. A61K 35/545; A61P 3/10; A61P 29/00; C12N 5/0735; C12N 5/074

(52) U.S. Cl. CPC A61K 35/545; A61P 3/10; A61P 29/00; C12N 5/0606; C12N 5/0696; C12N 2531/00

(57) ABSTRACT

The present disclosure provides a composition comprising extracellular vesicles (EVs) derived from induced pluripotent stem cells (iPSCs) or embryonic stem cells (ESCs). The present EVs possess immunoregulatory functions that are distinct from those of EVs derived from mesenchymal stem cells. The present EVs can be used to treat or modulate immune responses in diseases such as cancer or autoimmune diseases.

### RELATED APPLICATION

This application claims benefit of and priority to U.S. Provisional Patent Application Ser. No. 63/157,087, filed Mar. 5, 2021, which is incorporated herein by reference in its entirety.

### FIELD

The present disclosure relates generally to the field of extracellular vesicles (EVs). In one embodiment, provided herein are methods of making and uses of EVs from induced pluripotent stem cells (iPSCs) and embryonic stem cells (ESCs).

### BACKGROUND

One approach to modulate immune responses includes administration of mesenchymal stem cells (MSCs). MSCs have been reported to induce regulatory T cells (Treg) formation and suppress proliferation of CD8⁺ T cells, B cells, and NK cells, or to inhibit differentiation and maturation of antigen-presenting cells (APCs), including dendritic cells (DCs) and macrophages. Although the exact mechanisms underlying the immunomodulatory functions of MSCs remain largely unknown, accumulating evidence indicates that the beneficial effects of MSCs are not dependent on direct cell-cell interactions but mostly on paracrine factors secreted by these cells. In addition to MSCs-derived growth factors and cytokines, extracellular vesicles (EVs, also called exosomes) have been increasingly recognized as important players in the MSC immunosuppressive activity. EVs are lipid membrane-enclosed vesicles secreted by cells with diameter ranging from about 30 nm to 1000 nm that contain nucleic acids and proteins derived from the cell of origin. EVs are able to shuttle their content into target cells. The use of EVs as therapeutic agents instead of their parental cells could avoid many concerns caused by cell transplantation including the possibility of tumorigenesis, low cell survival or loss of function affected by the microenvironment. However, isolation of EVs from MSCs still has many drawbacks. MSCs are heterogeneous cell population without clearly defined specific markers and as such, their potency and consequently the potency of EVs might differ depending on the source of MSC, the age of the donor or culture conditions. MSCs can only be passaged a limited number of times and many studies have demonstrated that long-term expansion of MSCs could induce senescence that is associated with growth arrest and cell apoptosis as well as with a reduction of their regenerative properties.

Thus, there is a need for improved method of EVs production and for methods of modulating immune response by administering to a subject EVs isolated from a better source.

### SUMMARY

In one embodiment, the present disclosure provides a method for increased production of EVs. In another embodiment, the present disclosure provides a method for modulation of immune response by administering to a subject EVs isolated from induced pluripotent stem cells (iPSCs). iPSCs can be derived from adult somatic cells such as skin fibroblasts or peripheral blood mononuclear cells (PBMCs) by genetic reprograming or ‘forced’ introduction of reprogramming genes (e.g. Oct4, Sox2, Klf4 and c-Myc). These cells are reprogrammed back into an embryonic-like pluripotent state that enables development of an unlimited source of any type of human cells needed for therapeutic purposes. These cells can be produced from patients' own cells by a simple procedure (e.g., punch) and then reprogrammed in the lab using defined factors. EVs derived from iPSCs can be isolated from medium conditioned by iPSCs grown in static cultures or in a bioreactor, e.g. stirred tank bioreactor or orbital shaker (referred herein as dynamic culture). Results presented herein indicate that mechanical stimulation of cells in the bioreactor increases EV production. For example, in one embodiment, it was observed that EV secretion by iPSCs or ESCs was increased when the cells were cultured in a spinner flask bioreactor. In one embodiment, conditioned medium containing EVs can be collected after 1 day and EVs can be isolated by density gradient ultracentrifugation, differential ultracentrifugation, size-exclusion chromatography, affinity chromatography or precipitation. In one embodiment, about 2-3 times more iPSC EVs were isolated from the bioreactor culture compared to standard static culture (e.g., static iPSC EVs: 2.5 mg/l million cells/day, dynamic iPSC EVs: 5 mg/l million cells/day determined by Micro BCA assay). In some embodiments, EVs isolated both from dynamic or static cultures have similar size of around 150 nm in diameter. iPSCs or ESCs in bioreactor do not need to be cultured on microcarrier beads but they might grow as spheroids. Thus, this method for increased production of EVs would also be useful for 3D cultures of spheroids or organoids.

In one embodiment, the present disclosure provides a method for increased production of EVs from embryonic stem cells (ESCs). In another embodiment, the present disclosure provides a method for modulation of immune response by administering to a subject EVs isolated from embryonic stem cells (ESCs). EVs derived from ESCs can be isolated from medium conditioned by ESCs grown in static cultures or in a bioreactor, e.g. stirred tank bioreactor or orbital shaker (referred herein as dynamic culture). For example, in one embodiment, it was observed that EV secretion by ESCs was increased when the cells were cultured in a spinner flask bioreactor. In one embodiment, conditioned medium containing ESC-derived EVs can be collected after 1 day and EVs can be isolated by density gradient ultracentrifugation, differential ultracentrifugation, size-exclusion chromatography, affinity chromatography or precipitation. In one embodiment, about 2-3 times more ESC EVs were isolated from the bioreactor culture compared to standard static culture (static ESC EVs: 2.2 mg/l million cells/day, dynamic ESC EVs: 6 mg/l million cells/day determined by Micro BCA assay). ESCs in bioreactor do not need to be cultured on microcarrier beads but they might grow as spheroids. Thus, this method for increased production of ESC-derived EVs would also be useful for 3D cultures of spheroids or organoids.

Embryonic stem cells (ESCs) are cells derived from the inner cell mass of the blastocyst prior to implantation. They are pluripotent and have an unlimited capacity for self-renewal and the ability to differentiate into any somatic cell type. There are reports showing that ESC EVs can rejuvenate senescent cells or enhance cardiac function and tissue regeneration, however, so far the effect of ESC EVs on activation and polarization of immune cells has not been documented yet.

Multiple studies have reported that EVs from MSCs mimic the immunoregulatory function and regenerative capacity of MSCs. Results presented herein indicate that EVs isolated from iPSCs or ESCs have superior immunomodulatory properties compared to MSC EVs. Addition of iPSC EVs or ESC EVs to T cell culture suppressed T cell activation in a dose dependent manner and significantly increased formation of regulatory T cells. iPSC EVs and ESC EVs could also alter monocyte/macrophage cytokine secretion. Monocytes/macrophages cultured in the presence of iPSC EVs or ESC EVs secreted higher levels of anti-inflammatory cytokines such as IL-10 and decreased levels of inflammatory cytokines such TNF-α. T cells cultured in the presence of iPSC EVs secreted higher levels of anti-inflammatory cytokines such as IL-10 and decreased levels of inflammatory cytokines such as IFN-γ. In all the experiments presented herein, iPSC EVs were more powerful as compared to MSC EVs.

In one embodiment, the present disclosure provides a method for large-scale production of iPSC EVs and ESC EVs and their uses as modulators of immune response. As such, the iPSC or ESC EVs can be used as therapeutic agents in a wide variety of diseases in which it is necessary to suppress inflammation. The immunomodulatory properties of the iPSC and ESC EVs would also help in tissue regeneration or decrease the risk of transplant rejection.

In one embodiment, the present disclosure provides a composition comprising extracellular vesicles (EVs) derived from induced pluripotent stem cells (iPSCs). In one embodiment, the present disclosure provides a composition comprising extracellular vesicles (EVs) derived from embryonic stem cells (ESCs).

In one embodiment, the present disclosure provides a method of producing extracellular vesicles (EVs) derived from induced pluripotent stem cells (iPSCs) or embryonic stem cells (ESCs), comprising the steps of culturing the iPSCs or ESCs in spinner flask bioreactor, orbital shaker or stirred bioreactor; and collecting the EVs from culture medium of the iPSCs or ESCs.

Thus, in one aspect, composition is provided comprising extracellular vesicles (EVs) derived from induced pluripotent stem cells (iPSCs) or embryonic stem cells (ESCs). In some embodiments, the iPSCs are derived from fibroblasts, peripheral blood mononuclear cells, myoblasts, keratinocytes, melanocytes, hepatocytes, β-cells, dental pulp cells, blood cells, urine-derived renal epithelial cells, amniotic fluid stem cells, muscle cells, or adult stem cells.

In some embodiments, the iPSCs or ESCs are cultured in spinner flask bioreactor, orbital shaker or stirred bioreactor. In some embodiments, the iPSCs or ESCs are cultured on microcarrier beads or as 3D spheroids. In some embodiments, the microcarrier beads are about 80 to about 400 μm in diameter. In some embodiments, the spinner flask bioreactor or stirred bioreactor or orbital shaker is spun or stirred at about 20-500 rpm.

In some embodiments, the EVs regulate the activation and differentiation of macrophages. In some embodiments, the EVs promote differentiation of macrophages into M2 macrophages. In some embodiments, the EVs suppress secretion of inflammatory cytokines by macrophages. In some embodiments, the inflammatory cytokines comprise TNF-alpha. In some embodiments, the EVs promote secretion of anti-inflammatory cytokines by macrophages. In some embodiments, the anti-inflammatory cytokines comprise IL-10. In some embodiments, the EVs regulate the activation and differentiation of T cells. In some embodiments, the EVs promote differentiation of T cells into regulatory T cells. In some embodiments, he EVs suppress secretion of inflammatory cytokines by T cells. In some embodiments, the inflammatory cytokines comprise IFN-gamma. In some embodiments, the EVs promote secretion of anti-inflammatory cytokines by T cells. In some embodiments, the anti-inflammatory cytokines comprise IL-10.

In some embodiments, the EV composition further comprises a hydrogel composition. In some embodiments, the hydrogel composition comprises laponite/hyaluronic acid, a self-assembly polymer such as a bi-block or tri-block polymer, a self-assembly peptides, an optionally hydrophobically-modified biopolymer such as chitosan, hyaluronic acid, and alginate, or any combination thereof.

In some embodiments, a method is provided for producing extracellular vesicles (EVs) derived from induced pluripotent stem cells (iPSCs) or embryonic stem cells (ESCs), comprising the steps of
(i) culturing the iPSCs or ESCs by dynamic culture such as by spinner flask bioreactor, stirred tank bioreactor or orbital shaker; and
(ii) collecting the EVs from culture medium of the iPSCs or ESCs.
   In some embodiments, the iPSCs are derived from fibroblasts, peripheral blood mononuclear cells, myoblasts, keratinocytes, melanocytes, hepatocytes, β-cells, dental pulp cells, blood cells, urine-derived renal epithelial cells, amniotic fluid stem cells, muscle cells, or adult stem cells. In some embodiments, the iPSCs or ESCs are cultured on microcarrier beads or as 3D spheroids. In some embodiments, the microcarrier beads are about 80 to about 400 μm in diameter. In some embodiments, the spinner flask bioreactor or stirred bioreactor or orbital shaker is spun or stirred at about 20 to about 500 rpm.

In one aspect, a method is provided for treating an autoimmune disease or a disease characterized by increased inflammatory cytokine production, the method comprising administering to a subject in need thereof extracellular vesicles (EVs) derived from induced pluripotent stem cells (iPSCs) or embryonic stem cells (ESCs). In some embodiments, the iPSCs are derived from fibroblasts, peripheral blood mononuclear cells, myoblasts, keratinocytes, melanocytes, hepatocytes, β-cells, dental pulp cells, blood cells, urine-derived renal epithelial cells, amniotic fluid stem cells, muscle cells, or adult stem cells. In some embodiments, the iPSCs or ESCs are cultured in dynamic culture such as in a spinner flask bioreactor, stirred bioreactor or orbital shaker. In some embodiments, the spinner flask bioreactor or stirred tank bioreactor or orbital shaker is spun or stirred at about 20-500 rpm. In some embodiments, the iPSCs or ESCs are cultured on microcarrier beads or as 3D spheroids.

In some embodiments, the EVs promote secretion of anti-inflammatory cytokines by macrophages. In some embodiments, the anti-inflammatory cytokines comprise IL-10. In some embodiments, the EVs regulate the activation and differentiation of T cells. In some embodiments, the EVs promote differentiation of T cells into regulatory T cells. In some embodiments, the EVs suppress secretion of inflammatory cytokines by T cells. In some embodiments, the inflammatory cytokines comprise IFN-gamma. In some embodiments, the EVs promote secretion of anti-inflammatory cytokines by T cells. In some embodiments, the anti-inflammatory cytokines comprise IL-10.

In some embodiments, the EVs are provided in a hydrogel composition. In some embodiments, the hydrogel composition comprises hyaluronic acid, a di-block copolymer, a tri-block co-polymer, laponite, a self-assembly peptide, an optionally hydrophobically-modified biopolymers such as chitosan, hyaluronic acid, and alginate, or any combination thereof.

In some embodiments, the autoimmune disease is type I diabetes, rheumatoid arthritis, psoriasis, Crohn's disease, periodontitis, gingivitis, asthma, Alzheimer's disease, Parkinson's disease, systemic lupus erythematosus, multiple sclerosis, or protection of tissue graft after transplantation (for example, beta cells made by differentiation of iPSCs or embryonic stem cells). In some embodiments, the disease characterized by increased inflammatory cytokine production is septic shock, cytokine storm, or Graft-Versus-Host Disease.

In one aspect, a composition is provided comprising extracellular vesicles (EVs) in a hydrogel matrix wherein the EVs are derived from induced pluripotent stem cells (iPSCs) or embryonic stem cells (ESCs). In some embodiments, the iPSCs are derived from fibroblasts, peripheral blood mononuclear cells, myoblasts, keratinocytes, melanocytes, hepatocytes, O-cells, dental pulp cells, blood cells, urine-derived renal epithelial cells, amniotic fluid stem cells, muscle cells, or adult stem cells. In some embodiments, the iPSCs or ESCs are cultured in dynamic culture such as in a spinner flask bioreactor or stirred tank bioreactor or orbital shaker. In some embodiments, the iPSCs or ESCs are cultured on microcarrier beads or as 3D spheroids. In some embodiments, the microcarrier beads are about 80 to about 400 μm in diameter. In some embodiments, the spinner flask bioreactor or stirred bioreactor or orbital shaker is spun or stirred at 20-500 rpm. In some embodiments, the hydrogel composition comprises hyaluronic acid, self-assembly polymer such as a di-block copolymer, a tri-block copolymer, laponite, a self-assembly peptide, an optionally hydrophobically-modified biopolymer such as chitosan, hyaluronic acid, and alginate, or any combination thereof. In some embodiments, the concentration of EVs in the composition is about 0.05 mg to about 2.5 mg per mL of hydrogel. In some embodiments, the composition comprises 0-85% hyaluronic acid and 5-30% Pluronic F147. In some embodiments, the composition comprises 85% hyaluronic acid and 15% Pluronic F147. In some embodiments, the composition comprises 0.5-5% chitosan and 1-5% laponite. In some embodiments, the composition comprises 1% chitosan and 2% laponite. In some embodiments, the composition comprises 0.5-5% alginate and 10-200 mM CaSO4. In some embodiments, the composition comprises 2% alginate and 25 mM CaSO4.

These and other aspects of the invention will be appreciated from the ensuing descriptions of the figures and detailed description of the invention.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

FIGS. 1A-1I show characterization of mouse and human iPSCs and their growth under dynamic culture conditions. FIG. 1A shows static cultures of mouse iPSCs grown in the 2i serum-free medium and human iPSCs grown in mTeSR Plus or E8 media FIG. 1B shows immunofluorescence staining of stem cell markers such as Oct4 and Sox2 for mouse and human iPSCs. FIG. 1C shows cell culture on an orbital shaker and in a spinner flask bioreactor. FIG. 1D shows the effects of different agitation rates on the growth of iPSCs. FIG. 1E shows homogenous distribution of cells in the spheroids under dynamic culture conditions. In static cultures, cells cluster randomly and form aggregates of variable sizes. FIG. 1F shows the area of spheroids obtained from the static or dynamic culture. FIG. 1G shows the circularity of spheroids grown under static and dynamic conditions. FIG. 1H shows mouse and human iPSC spheroid growth in a bioreactor over period of 3 days. FIG. 1I shows comparison of sizes of mouse and human iPSC spheroids grown in a bioreactor on days 1, 2, and 3.

FIGS. 2A-2F show increased production of extracellular vesicles by dynamic culture on an orbital shaker and in a spinner flask bioreactor. FIG. 2A shows Western blot of iPSC and MSC cells (C) and extracellular vesicles (E). EV isolations do not contain calnexin (CALNX, a marker of endoplasmic reticulum but they are positive for EV markers such as CD63, CD81 and Flotillin (Flot). FIG. 2B shows representative transmission electron microscopy image of EVs isolated from human iPSCs. FIG. 2C shows flow cytometry analysis of human iPSC and MSC EVs and ESC EVs positive for EV markers CD9, CD81, and CD63. Peaks are, left to right, Blank, MSC EVs, ESC EVs and iPSC EVs. FIG. 2D shows increased production of EVs by dynamically cultured iPSCs. FIG. 2E shows comparison of concentrations of EVs isolated from iPSC static or dynamic culture in a spinner flask bioreactor. FIG. 2F shows comparison of the mode of EV sizes of EVs obtained from the static or dynamic cultures.

FIGS. 3A-3E show mass spectroscopy assisted characterization of EVs from iPSCs and MSCs. FIG. 3A shows the overlap of proteins detected in EVs obtained from static and dynamic cultures of iPSCs. FIG. 3B shows identified EV surface markers. FIG. 3C shows the origins of the different proteins identified in the iPSC EVs. FIGS. 3D and 3E compare the proportions of different classes of proteins in iPSC EVs vs MSC EVs. For each component or process, the upper bar represents iPSC EVs and the lower, MSC EVs.

FIGS. 4A-4D show an embodiment of hydrogel-encapsulated EVs. In situ forming hydrogel for EV delivery can be based on polymers such as laponite, hyaluronic acid (HA), and Pluronic F127 (Poloxamer 407). FIG. 4A shows schematic of EVs in laponite hydrogel. FIG. 4B shows the release of EVs over time from laponite hydrogel or control alginate hydrogel. FIG. 4C shows the sol-gel transition behavior of HA (1 wt/v %) and Pluronic F127 (15 wt/v %) with the gelation temperature (Tgel) of 29° C. FIG. 4D shows cumulative release of iPSC EVs from HA and Pluronic gel in vitro over the period of 7 days.

FIGS. 5A-5D show the effects of MSC EVs or iPSC EVs on the activation and differentiation of macrophages. FIG. 5A shows the expression of arginase on human monocytic THP-1 cells cultured in different conditions: M1: THP-1 cells cultured to become M1 macrophages; M2: THP-1 cells cultured to become M2 macrophages; MSC EV: THP-1 cells co-cultured with MSC EVs; iPSC EV: THP-1 cells co-cultured with iPSC EVs. FIG. 5B shows the expression of CD206 on human monocytic THP-1 cells cultured in different conditions as in FIG. 5A. FIG. 5C shows the production of TNF-α by THP-1 cells cultured in the presence or absence of MSC EVs or iPSC EVs. FIG. 5D shows the production of IL-10 by THP-1 cells cultured in the presence or absence of MSC EVs or iPSC EVs.

FIGS. 6A-6D show the effects of MSC EVs or iPSC EVs on the activation and differentiation of T cells. FIG. 6A shows the expression of CD25 (% CD25⁺; left panel) and CD44 (mean fluorescence intensity; right panel) in naïve CD4⁺ T cells primed with anti-CD3 and anti-CD28 in the absence (Ctrl) or presence of MSC EVs or iPSC EVs. FIG. 6B shows the production of IFN-γ and IL-10 by CD4⁺ T cells primed with anti-CD3 and anti-CD28 in the absence (Ctrl) or presence of MSC EVs or iPSC EVs. FIG. 6C shows the effects of MSC EVs or iPSC EVs on the induction of regulatory T cells (Treg). CD25 and Foxp3 co-expression was examined on naïve CD4⁺ T cells primed with anti-CD3 and anti-CD28 in the absence (Ctrl) or presence of MSC EVs or iPSC EVs. FIG. 6D shows the effect of mouse (miPSC EVs) and human iPSC (hiPSC) EVs (100 ug/ml) isolated from static and dynamic cultures on the induction of human regulatory T cells (Tregs) in the absence of TGF-β.

FIGS. 7A-7E show immunoregulatory properties of iPSC EVs in two mouse models of T1D: adoptive transfer model of T1D in NOD/scid mice and spontaneously developed T1D in NOD mice. FIG. 7A presents a toxicology profile on blood samples from control mice, mice treated with hydrogel only, or mice treated with hydrogel-encapsulated iPSC EVs. Each set of three bars shows, left to right, Control, Hydrogel only and iPSC EV+hydrogel. FIG. 7B shows endpoint measurement of blood glucose levels in adoptive transfer model of T1D into immunodeficient mice. In contrast to untreated mice and mice injected with hydrogel alone, mice treated with iPSC EVs+hydrogel had normal blood glucose levels at the end of the experiment. FIG. 7C shows ratio of FoxP3+ T cells to IFN-gamma producing cells in pancreases and spleens of untreated mice, mice treated with hydrogel only, or mice treated with hydrogel-encapsulated iPSC EVs. FIG. 7D shows the production of IFN-gamma, IL-2, and IL-10 by T cells from control mice (untreated), mice treated with hydrogel only, or mice treated with hydrogel-encapsulated iPSC EVs. In FIGS. 7B, C and D, each graph shows data for, left to right, Untreated, Hydrogel Only and iPSC EV+Hydrogel. FIG. 7E shows spontaneous development of TID in control (untreated) NOD mice, NOD mice treated with hydrogel alone but not in NOD mice that obtained two doses of iPSC EVs encapsulated in a hydrogel.

FIGS. 8A-8H show ESC dynamic culture, increased production of EV in a bioreactor and ESC EV immunomodulatory properties comparable to iPSC EVs. FIG. 8A shows homogeneous distribution of cells in the spheroids under dynamic culture conditions (40 rpm). FIG. 8B shows representative transmission electron microscopy image of EVs isolated from human ESCs with diameter around 200 nm. FIG. 8C shows increased production of EVs by dynamically cultured ESCs documented by increased concentrations of EVs from dynamic ESC culture in a spinner flask bioreactor. FIG. 8D shows similarity of protein composition between iPSC EVs and ESC EVs. FIG. 8E shows the origins of the different proteins identified in the ESC EVs. For each component, the upper bar represents iPSC EVs and the lower, ESC EVs. FIG. 8F shows the production of TNF-α by macrophages cultured in the absence (Ctrl) or presence of ESC EVs (left panel). Right panel shows the production of IL-10 by macrophages cultured in the absence (Ctrl) or presence of ESC EVs. FIG. 8G shows suppression of CD4⁺ T cell activation by ESC EVs. Naïve CD4⁺ T cell were primed with anti-CD3 and anti-CD28 in the absence (Ctrl) or presence of ESC EVs. FIG. 8H shows the effects of ESC EVs on the induction of regulatory T cells (Treg). CD25 and Foxp3 co-expression was examined in naïve CD4⁺ T cells primed with anti-CD3 and anti-CD28 in the absence (Ctrl) or presence of ESC EVs.

### DETAILED DESCRIPTION

In one embodiment, the present disclosure determined an optimized extracellular vesicle (EV; also called exosome) isolation method to achieve the highest EV yields. EV secretion under standard static culture conditions was compared with EV secretion under dynamic culture conditions (e.g., in spinner flask bioreactor), and it was found that dynamic culture conditions would lead to improved EV production.

In another embodiment, the present disclosure studied the immunomodulatory properties of EVs isolated from induced pluripotent stem cells (iPSC) and embryonic stem cells (ESCs) and compare them with the currently most studied MSC EVs. Results presented herein shed a light on the paracrine immunomodulatory function of iPSC EVs and ESC EVs that may be utilized in various bioengineering or immunoregulatory applications.

iPSCs can be derived from adult somatic cells such as skin fibroblasts or peripheral blood mononuclear cells (PBMCs) by genetic reprograming. Other cell types from which they are derivable include but are not limited to fibroblasts, myoblasts, keratinocytes, melanocytes, hepatocytes, 0-cells, dental pulp cells, blood cells, urine-derived renal epithelial cells, amniotic fluid stem cells, muscle cells, adult stem cells. The iPSCs are reprogrammed back into an embryonic-like pluripotent state and they should display theoretically unlimited proliferative capacity in culture. Currently, there are no reports on the effect of iPSC EVs on immune cells. The present disclosure examined the vesicular transcriptome and proteome to unveil the components that contribute to the immunomodulatory properties of EVs. The effects of isolated EVs on innate and adaptive immune cells were tested in vitro. Macrophages are generally reported to be among the first cells to internalize EVs when administered in the body. Therefore, the ability of iPSC EVs to affect macrophage polarization was tested and compared with MSC EVs. The effects of iPSC EVs on T cell activation/suppression and Treg formation were also analyzed. Lastly, the immunomodulatory properties of iPSC EVs can be evaluated in vivo, e.g., in the NOD mouse model of type 1 diabetes. For this purpose, a delivery platform was developed to ensure localized long-term release of EVs. In one embodiment, the delivery platform comprises hydrogel-encapsulated EVs. One of ordinary skill in the art would readily construct suitable hydrogel compositions according to techniques generally known in the art.

Embryonic stem cells (ESCs) are cells derived from the inner cell mass of the blastocyst prior to implantation. They are pluripotent and have an unlimited capacity for self-renewal and the ability to differentiate into any somatic cell type. Currently, there are no reports on the effect of ESC EVs on immune cells. The present disclosure examined the vesicular composition to unveil the components that contribute to the immunomodulatory properties of EVs. The effects of isolated EVs on innate and adaptive immune cells were tested in vitro. The ability of ESC EVs to affect macrophage polarization was comparable to iPSC EVs. The effects of ESC EVs on the suppression of activation of T cells and formation of Treg were also analyzed. Crosslinked hyaluronic acid (HA) can control release and bioavailability of encapsulated reagents, e.g., EVs.

Chemical crosslinking can affect the bioactivity of encapsulated protein-containing therapeutics. In one embodiment, hydrogels are used as the encapsulating agent. In one embodiment, biopolymers that comprise the hydrogel include but are not limited to chitosan, alginate and hyaluronic acid, or any combination thereof. In some embodiments the biopolymer is optionally hydrophobically modified. In some embodiments, the biopolymer or biopolymers are hydrophobically modified as described herein. In some embodiments, the hydrogel comprises hydrophobically modified biopolymers such as but not limited to alginate, chitosan and hyaluronic acid, or the combination thereof. In some embodiments the biopolymer or biopolymers are not hydrophobically modified. In some embodiments, hydrogels comprised of biopolymers that are not hydrophobically modified further comprise at least one cross-linking agent to provide the desired hydrogel properties. In some embodiments the crosslinking agent is a self-assembly polymer or a self-assembly peptide. Such hydrogel can be prepared by the addition of another polymer such as a di-block or tri-block copolymer that can self-assemble at, e.g., body temperature. In one embodiment, physically crosslinked hydrogels can be utilized as EV delivery platforms. In one embodiment, hydrogels comprise an optionally hydrophobically modified biopolymer. In one embodiment, hydrogels comprise an optionally hydrophobically modified biopolymer such as alginate, chitosan or hyaluronic acid.

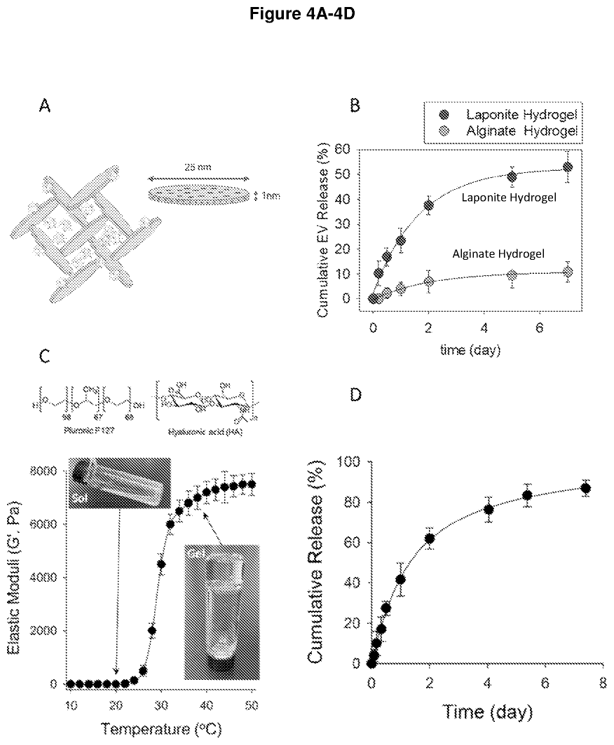

In one non-limiting example, such a self-assembly polymer called Pluronic is included in the HA formulation. Pluronics (PEOx-PPOy-PEOx) are non-ionic triblock copolymers composed of two hydrophilic side chains of poly (ethylene oxide), PEO, and a central hydrophobic chain of poly(propylene oxide), PPO. They are also known as poloxamers and are non-toxic and biodegradable. One of the main characteristics of Pluronic F127 is that it forms a gel at relatively low concentrations and temperatures close to room temperature, which is very useful for biomedical applications. However, Pluronic hydrogel structure is not maintained for a long time in physiological conditions, due to the low molecular weight and low mechanical strength, which result in burst drug release. Incorporation of HA can, in one embodiment, fix this issue as incorporated HA chains can bridge Pluronic micelles and stabilize the hydrogel to provide e.g., 1-2 weeks of sustained release. In other embodiments, other durations of release can be provided. We have demonstrated the feasibility of this approach (FIG. 4D).

In some embodiments, laponite is included in the hydrogel, which in some embodiments facilitates gelation. In some embodiments the laponite is Laponite XLG. In some embodiments laponite is include at about 1 to about 5%. In some embodiments laponite is include at about 2%.

In some embodiments, the loading of the hydrogel with EV is about 5-250 micrograms of EVs per 100 uL of hydrogel (e.g., about 50 ug to about 2.5 mg per mL of hydrogel). In some embodiments, the loading of the hydrogel with EV is about 25-75 micrograms of EVs per 100 uL of hydrogel. In some embodiments, the loading is about 40 micrograms of EVs per 100 uL of hydrogel. In some embodiments, the loading is about 100 micrograms of EVs per 100 uL of hydrogel. In some embodiments, the loading is about 50 micrograms of EVs per μL of hydrogel.

In some embodiments, the hydrogel comprises hyaluronic acid and self-assembly polymers such as Pluronic. In some embodiments, the hydrogel comprises about 85% hyaluronic acid and about 15% Pluronic F127.

In some embodiments the hydrogel comprises laponite, self-assembly peptides, optionally hydrophobically-modified biopolymers including chitosan, hyaluronic acid, and alginate.

Non-limiting examples of self-assembly peptides include the self-assembling peptide Q11 (QQKFQFQFEQQ; SEQ ID NO:1), which assembles into nanofibers and hydrogels under physiological conditions, and peptide amphiphiles. In some embodiments, the self-assembly peptide is a peptide amphiphile, comprising (i) a long alkyl tail that conveys hydrophobic character to the molecule and, when combined with the peptide region, makes the molecule amphiphilic; (ii) two-eight consecutive cysteine residues that when oxidized may form disulfide bonds to polymerize the self-assembled structure; (iii) a flexible linker region of two-eight glycine residues to provide the hydrophilic head group flexibility from the more rigid cross-linked region; and (iv) a peptide sequence, e.g. Arg-Gly-Asp (RGD) sequence which is contained collagen-associated protein and fibronectin. This sequence has been found to play an important role in integrin-mediated cell adhesion. Examples are provided in Y Wen, A Waltman, H Han, JH Collier, Switching the immunogenicity of peptide assemblies using surface properties, ACS Nano (2016) 9274-9286; JD Hartgerink, E Beniash, SI Stupp, Self-assembly and mineralization of peptide-amphiphile nanofibers, Science, 294 (2001) 684-1688; each of which is incorporated by reference herein in its entirety.

In some embodiments chitosan, the polymer used for the hydrogel is hyaluronic acid, chitosan or alginate. In some embodiments, the polymer used for the hydrogel is hydrophobically modified. In some embodiments chitosan, hyaluronic acid or alginate are hydrophobically modified. Hydrophobic modifications of polymers is known in the art. For example, hydrophobic modification of chitosan is achieved by dissolving one gram chitosan (e.g., CS; medium molecular weight, 280,000 g/mol, degree of deacetylation 83%, Fluka) in aqueous acetic acid (50 mL, 1% w/v; Sigma-Aldrich). Once dissolved, the solution is filtered with a 0.45 μm Nylon syringe filter. The pH is then adjusted to 5.5 by addition of sodium hydroxide (Sigma-Aldrich). A solution of 300 mg palmitic acid N-hydroxysuccinimide ester (Sigma-Aldrich) in absolute ethanol (Sigma-Aldrich) is added drop-wise to the chitosan solution at 98° C. under reflux and reacted for 48 h. The solution is then cooled down to room temperature and, after adding acetone, is precipitated by adjusting pH of the solution to 9.0. The precipitated polymer is then filtered twice, washed with an excess of acetone, and vacuum-dried at room temperature. Other non-limiting methods for preparing hydrophobically modified chitosan are found in Majedi F. S., Hasani-Sadrabadi M. M., VanDersarl J. J., Mokarram N., Hojjati Emami S., Dashtimoghadam E., Bertsch A., Renaud, P. On-Chip Fabrication of Paclitaxel Loaded Chitosan Nanoparticles for Cancer Therapeutics. Advanced Functional Materials. 24 (2014) 432-441; Majedi, F. S., Hasani-Sadrabadi M. M., Emami, S. H., Shokrgozar, M. A., VanDersal, J. J., Dashtimoghadam, E., Bertsch, A., Moaddel, H., Renaud, P., Microfluidic Assisted Self-assembly of Chitosan based Nanoparticles as Drug Delivery Agents. Lab on a Chip 13 (2013) 204-207; YL Chiu, SC Chen, CJ Su, CW Hsiao, YM Chen, pH-triggered injectable hydrogels prepared from aqueous N-palmitoyl chitosan: In vitro characteristics and in vivo biocompatibility, Biomaterials. 30 (2009) 4877-4888; each of which is incorporated herein by reference in its entirety. Any of such methods may be used to modify other biopolymers used in the hydrogels disclosed herein, such as but not limited to alginate and hyaluronic acid.

In some embodiments, cross-linking of the hydrogel provides controlled release properties. In some embodiments, the hydrogel releases EVs over 1-7 days. In some embodiments the hydrogel releases EVs over 1-2 weeks. In some embodiments, at least two compositions are administered, one providing an immediate release of EVs and the other providing controlled release. The hydrogel is provided, in some embodiments, for administration parenterally such as but not limited to subcutaneously, intradermally, intravenously, intramuscularly, topically, nasally, intraperitoneally, or implanted in any organ or tissue.

The foregoing examples of hydrogel components are provided as a guide for preparing the EVs disclosed herein for administration to achieve the purposes disclosed herein. In one embodiment, an optimal hydrogel formulation for EV delivery is based on an in-situ forming hydrogel with sol-to-gel transition that can be triggered by changing the pH or temperature to physiological conditions. Such a formulation forms a depot and allow sustained release of EVs for extended period of time.

Non-limiting examples of formulations of hydrogels for administering and delivering EVs include (values in wt % unless otherwise indicated):
0-85% hyaluronic acid, 5-30% Pluronic F147; example: 85% hyaluronic acid, 15% Pluronic F147;
0.5-5% chitosan, 1-5% laponite; example: 1% chitosan, 2% laponite;
0.5-5% alginate (10-200 mM CaSO4); example: 2% alginate (25 mM CaSO4).

In some embodiments, the hydrogel comprises 85% hyaluronic acid and 15% Pluronic F147. In some embodiments the hydrogel comprises 1% chitosan and 2% laponite. In some embodiments the hydrogel comprises 2% alginate (25 mM CaSO4). In some embodiments, the hydrogel consists of 85% hyaluronic acid and 15% Pluronic F147. In some embodiments the hydrogel consists of 1% chitosan and 2% laponite. In some embodiments the hydrogel consists of 2% alginate (25 mM CaSO4). In any of the foregoing aspects, other components may be present such as but not limited to a buffer, salts, water, or culture medium components (e.g., the components of RMPI 1640).

The use of EVs secreted by iPSCs brings several advantages. The cells and consequently the EVs can be autologous, i.e. derived from patient's own cells but also allogenic iPSC EVs can be potentially used because they contain low levels of HLA (human leukocyte antigens). The EVs can also be derived from genetically modified pluripotent stem cells to be more immunocompatible, thereby making the EV production much more cost effective. Other advantages of EV isolation from iPSCs is that the iPSC are highly expandable and reproducible and can be cultured in serum-free medium, which eliminates the risk of EV contamination with vesicles from serum that is frequently used as a supplement of different cell culture media.

Similarly to iPSCs, ESCs are also highly expandable and reproducible and can be cultured in serum-free medium, which eliminates the risk of EV contamination with vesicles from serum that is frequently used as a supplement of different cell culture media. These cells grow very fast, which enables rapid cell expansion for EV production. Moreover, ESCs or iPSCs are frequently cultured in large scale and then differentiated into different cell types for transplantations (such as beta cells, cardiomyocytes) and EVs as the byproducts of the stem cell expansion phase can be collected and used to achieve better cell survival and protection of the differentiated cells after transplantation into the body.

In view of the immunoregulatory functions of iPSC EVs and ESC EVs disclosed herein, one would readily apply the iPSC EVs and ESC EVs in various immunoregulatory applications. For example, the iPSC EVs and ESC EVs can be used to treat or modulate immune responses in diseases such as virus-induced diseases, autoimmune diseases, immune disorder, cancer, or combination thereof.

In one embodiment, a viral infection is caused by viruses in the Baltimore classification Group I group of viruses: double-stranded DNA viruses (e.g. Adenoviruses, Herpesviruses, Poxviruses). In another embodiment, the viral infection is caused by viruses in the Baltimore classification Group II group of viruses: single-stranded (or “sense”) DNA viruses (e.g. Parvoviruses). In another embodiment, the viral infection is caused by viruses in the Baltimore classification Group III group of viruses: double-stranded RNA viruses (e.g. Reoviruses). In another embodiment, the viral infection is caused by viruses in the Baltimore classification Group IV group of viruses: single-stranded (sense) RNA viruses (e.g. Picornaviruses, Togaviruses, Coronavirus). In another embodiment, the viral infection is caused by viruses in the Baltimore classification Group V of viruses: single-stranded (antisense) RNA viruses (e.g. Orthomyxoviruses, Rhabdoviruses). In another embodiment, the viral infection is caused by viruses in the Baltimore classification Group VI group of viruses: single-stranded (sense) RNA viruses with DNA intermediate in life-cycle (e.g. Retroviruses). In another embodiment, the viral infection is caused by viruses in the Baltimore classification Group VII group of viruses: double-stranded DNA viruses with RNA intermediate in life-cycle (e.g. Hepadnaviruses).

In one embodiment, the virus-induced disease can be respiratory viral infection (e.g. common cold, seasonal influenzas), gastrointestinal viral infection, liver viral infection, nervous system viral infection, skin viral infection, sexually transmitted viral infection, placental viral infection, or fetal viral infection.

In one embodiment, representative examples of autoimmune disease include, but are not limited to, achalasia, amyloidosis, ankylosing spondylitis, anti-gbm/anti-tbm nephritis, antiphospholipid syndrome, arthritis, autoimmune angioedema, autoimmune encephalomyelitis, autoimmune hepatitis, autoimmune myocarditis, autoimmune oophoritis, autoimmune orchitis, autoimmune pancreatitis, autoimmune retinopathy, autoimmune urticaria, Behcet's disease, celiac disease, chagas disease, chronic inflammatory demyelinating polyneuropathy, Cogan's syndrome, congenital heart block, Crohn's disease, dermatitis, dermatomyositis, discoid lupus, Dressler's syndrome, endometriosis, fibromyalgia, fibrosing alveolitis, granulomatosis with polyangiitis, Graves' disease, Guillain-Barre syndrome, herpes gestationis, immune thrombocytopenic purpura, interstitial cystitis, juvenile arthritis, juvenile diabetes (type 1 diabetes), juvenile myositis, Kawasaki disease, Lambert-Eaton syndrome, lichen planus, lupus, Lyme disease, multiple sclerosis, myasthenia gravis, myositis, neonatal lupus, neutropenia, palindromic rheumatism, peripheral neuropathy, polyarteritis nodosa, polymyalgia rheumatica, polymyositis, postmyocardial infarction syndrome, postpericardiotomy syndrome, primary biliary cirrhosis, primary sclerosing cholangitis, progesterone dermatitis, psoriasis, psoriatic arthritis, reactive arthritis, retroperitoneal fibrosis, rheumatic fever, rheumatoid arthritis, sarcoidosis, Schmidt syndrome, scleritis, scleroderma, Sjagren's syndrome, thrombocytopenic purpura, periodontitis, gingivitis, asthma, Alzheimer's disease, Parkinson's disease, systemic lupus erythematosus, protection of tissue graft after transplantation (for example, beta cells made by differentiation of iPSC or embryonic stem cells), ulcerative colitis, uveitis, vasculitis, and vitiligo.

In one embodiment, representative examples of cancer include, but are not limited to, carcinoma, sarcoma, lymphoma, leukemia, germ cell tumor, blastoma, chondrosarcoma, Ewing's sarcoma, malignant fibrous histiocytoma of bone, osteosarcoma, rhabdomyosarcoma, heart cancer, brain cancer, astrocytoma, glioma, medulloblastoma, neuroblastoma, breast cancer, medullary carcinoma, adrenocortical carcinoma, thyroid cancer, Merkel cell carcinoma, eye cancer, gastrointestinal cancer, colon cancer, gallbladder cancer, gastric (stomach) cancer, gastrointestinal carcinoid tumor, hepatocellular cancer, pancreatic cancer, rectal cancer, bladder cancer, cervical cancer, endometrial cancer, ovarian cancer, renal cell carcinoma, prostate cancer, testicular cancer, urethral cancer, uterine sarcoma, vaginal cancer, head cancer, neck cancer, nasopharyngeal carcinoma, hematopoietic cancer, Non-Hodgkin lymphoma, skin cancer, basal-cell carcinoma, melanoma, small cell lung cancer, non-small cell lung cancer, or any combination thereof.

The terms “comprise”, “comprises”, “comprising”, “includes”, “including”, “having” and their conjugates mean “including but not limited to”.

As used herein, the singular form “a”, “an” and “the” include plural references unless the context clearly dictates otherwise. For example, the term “an enzyme” or “at least one enzyme” may include a plurality of enzymes, including mixtures thereof.

Throughout this application, various embodiments of the present disclosure may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

When values are expressed as approximations, by use of the antecedent “about,” it is understood that the particular value forms another embodiment. All ranges are inclusive and combinable. In one embodiment, the term “about” refers to a deviance of between 0.1-5% from the indicated number or range of numbers. In another embodiment, the term “about” refers to a deviance of between 1-10% from the indicated number or range of numbers. In another embodiment, the term “about” refers to a deviance of up to 20% from the indicated number or range of numbers. In one embodiment, the term “about” refers to a deviance of ±10% from the indicated number or range of numbers. In another embodiment, the term “about” refers to a deviance of ±5% from the indicated number or range of numbers.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting. Each literature reference or other citation referred to herein is incorporated herein by reference in its entirety.

In the description presented herein, each of the steps of the invention and variations thereof are described. This description is not intended to be limiting and changes in the components, sequence of steps, and other variations would be understood to be within the scope of the present invention.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the terms “treat”, “treatment”, or “therapy” (as well as different forms thereof) refer to therapeutic treatment, including prophylactic or preventative measures, wherein the object is to prevent or slow down (lessen) an undesired physiological change associated with a disease or condition. Beneficial or desired clinical results include, but are not limited to, alleviation of symptoms, diminishment of the extent of a disease or condition, stabilization of a disease or condition (i.e., where the disease or condition does not worsen), delay or slowing of the progression of a disease or condition, amelioration or palliation of the disease or condition, and remission (whether partial or total) of the disease or condition, whether detectable or undetectable. Those in need of treatment include those already with the disease or condition as well as those prone to having the disease or condition or those in which the disease or condition is to be prevented.

As used herein, the terms “component,” “composition,” “formulation”, “composition of compounds,” “compound,” “drug,” “pharmacologically active agent,” “active agent,” “therapeutic,” “therapy,” “treatment,” or “medicament,” are used interchangeably herein, as context dictates, to refer to a compound or compounds or composition of matter which, when administered to a subject (human or animal) induces a desired pharmacological and/or physiologic effect by local and/or systemic action. A personalized composition or method refers to a product or use of the product in a regimen tailored or individualized to meet specific needs identified or contemplated in the subject.

As used herein, “modulating” refers to “stimulating” or “inhibiting” an activity of a molecular target or pathway. For example, a composition modulates the activity of a molecular target or pathway if it stimulates or inhibits the activity of the molecular target or pathway by at least 10%, by at least about 20%, by at least about 25%, by at least about 30%, by at least about 40%, by at least about 50%, by at least about 60%, by at least about 70%, by at least about 75%, by at least about 80%, by at least about 90%, by at least about 95%, by at least about 98%, or by about 99% or more relative to the activity of the molecular target or pathway under the same conditions but lacking only the presence of the composition. In another example, a composition modulates the activity of a molecular target or pathway if it stimulates or inhibits the activity of the molecular target or pathway by at least 2-fold, at least 5-fold, at least 10-fold, at least 20-fold, at least 50-fold, at least 100-fold relative to the activity of the molecular target or pathway under the same conditions but lacking only the presence of the composition. The activity of a molecular target or pathway may be measured by any reproducible means. The activity of a molecular target or pathway may be measured in vitro or in vivo. For example, the activity of a molecular target or pathway may be measured in vitro or in vivo by an appropriate assay known in the art measuring the activity. Control samples (untreated with the composition) can be assigned a relative activity value of 100%.

The terms “subject,” “individual,” and “patient” are used interchangeably herein, and refer to human or non-human animals to whom treatment with a composition or formulation in accordance with the present invention is provided. The terms “non-human animals” and “non-human mammals” are used interchangeably herein and include all vertebrates, e.g., mammals, such as non-human primates (e.g. higher primates), sheep, dog, rodent (e.g. mouse or rat), guinea pig, goat, pig, cat, rabbits, cows, horses, or non-mammals such as reptiles, amphibians, chickens, and turkeys. The compositions described herein can be used to treat any suitable mammal, including primates, such as monkeys and humans, horses, cows, cats, dogs, rabbits, and rodents such as rats and mice. In one embodiment, the mammal to be treated is human. The human can be any human of any age. In one embodiment, the human is an adult. In another embodiment, the human is a child. The human can be male, female, pregnant, middle-aged, adolescent, or elderly.

Pharmaceutical compositions suitable for use in the methods disclosed herein include compositions wherein the active ingredients are contained in an amount effective to achieve the intended purpose. In one embodiment, a therapeutically effective amount means an amount of active ingredients effective to prevent, alleviate or ameliorate symptoms of disease or prolong the survival of the subject being treated. Determination of a therapeutically effective amount is well within the capability of those skilled in the art.

In one embodiment, the present disclosure provides a composition comprising extracellular vesicles (EVs) derived from induced pluripotent stem cells (iPSCs). The iPSCs can be derived from any suitable cell types, e.g. the iPSCs are derived from fibroblasts or peripheral blood mononuclear cells. In some embodiments, the iPSCs are derived from fibroblasts, PBMCs, myoblasts, keratinocytes, melanocytes, hepatocytes, β-cells, dental pulp cells, blood cells, urine-derived renal epithelial cells, amniotic fluid stem cells, muscle cells, adult stem cells. In one embodiment, the iPSCs are cultured in spinner flask bioreactor or stirred bioreactor. The present EVs can regulate the activation and differentiation of macrophages. In one embodiment, the EVs promote differentiation of macrophages into M2 macrophages. In another embodiment, the EVs suppress secretion of inflammatory cytokines by macrophages. Examples of inflammatory cytokines include, but are not limited to, TNF-alpha. In another embodiment, the EVs promote secretion of anti-inflammatory cytokines by macrophages. Examples of anti-inflammatory cytokines include, but are not limited to, IL-10.

In one embodiment, the present disclosure provides a composition comprising extracellular vesicles (EVs) derived from embryonic stem cells (ESCs).

In another embodiment, the present EVs can regulate the activation and differentiation of T cells. In one embodiment, the EVs promote differentiation of T cells into regulatory T cells. In another embodiment, the EVs suppress secretion of inflammatory cytokines by T cells. Examples of inflammatory cytokines include, but are not limited to, IFN-gamma. In another embodiment, the EVs promote secretion of anti-inflammatory cytokines by T cells. Examples of anti-inflammatory cytokines include, but are not limited to, IL-10.

In one embodiment, the above EV composition further comprises a hydrogel composition. For example, the hydrogel composition comprises laponite.

In one embodiment, the present disclosure provides a method of producing extracellular vesicles (EVs) derived from induced pluripotent stem cells (iPSCs), comprising the steps of culturing the iPSCs in a dynamic culture such as but not limited to spinner flask bioreactor, orbital shaker or stirred tank bioreactor; and collecting the EVs from culture medium of the iPSCs. The iPSCs can be derived from any suitable cell types, e.g. the iPSCs are derived from fibroblasts peripheral blood mononuclear cells, myoblasts, keratinocytes, melanocytes, hepatocytes, β-cells, dental pulp cells, blood cells, urine-derived renal epithelial cells, amniotic fluid stem cells, muscle cells, adult stem cells, by way of non-limiting examples.

In one embodiment, the present disclosure provides a method of producing extracellular vesicles (EVs) derived from embryonic stem cells (ESCs), comprising the steps of culturing the iPSCs in a dynamic culture such as but not limited to spinner flask bioreactor, orbital shaker or stirred tank bioreactor; and collecting the EVs from culture medium of the ESCs.

The following numbered embodiments, while non-limiting, are exemplary of certain aspects of the disclosure:
1. A composition comprising extracellular vesicles (EVs) derived from induced pluripotent stem cells (iPSCs).
2. The composition of embodiment 1, wherein the iPSCs are derived from adult somatic cells.
3. The composition of embodiment 2, wherein the adult somatic cells are fibroblasts or peripheral blood mononuclear cells.
4. The composition of embodiment 1, wherein the iPSCs are cultured in spinner flask bioreactor, orbital shaker, or stirred bioreactor.
5. The composition of embodiment 1, wherein the iPSCs are cultured as 3D spheroids.
6. The composition of embodiment 4, wherein the spinner flask bioreactor, stirred bioreactor or orbital shaker is spun or stirred at a speed from about 30 rpm to about 300 rpm.
7. The composition of embodiment 1, wherein the EVs regulate the activation and differentiation of macrophages.
8. The composition of embodiment 7, wherein the EVs promote differentiation of macrophages into M2 macrophages.
9. The composition of embodiment 7, wherein the EVs suppress secretion of inflammatory cytokines by macrophages.
10. The composition of embodiment 9, wherein the inflammatory cytokines comprise TNF-α.
11. The composition of embodiment 7, wherein the EVs promote secretion of anti-inflammatory cytokines by macrophages.
12. The composition of embodiment 11, wherein the anti-inflammatory cytokines comprise IL-10.
13. The composition of embodiment 1, wherein the EVs regulate the activation and differentiation of T cells.
14. The composition of embodiment 13, wherein the EVs promote differentiation of T cells into regulatory T cells.
15. The composition of embodiment 13, wherein the EVs suppress secretion of inflammatory cytokines by T cells.
16. The composition of embodiment 15, wherein the inflammatory cytokines comprise IFN-γ.
17. The composition of embodiment 13, wherein the EVs promote secretion of anti-inflammatory cytokines by T cells.
18. The composition of embodiment 17, wherein the anti-inflammatory cytokines comprise IL-10.
19. The composition of embodiment 1, further comprising a hydrogel composition.
20. The composition of embodiment 19, wherein the hydrogel composition comprises laponite.
21. The composition of embodiment 19, wherein the hydrogel composition further comprises chitosan.
22. A method of producing extracellular vesicles (EVs) derived from induced pluripotent stem cells (iPSCs), comprising the steps of
   (iii) culturing the iPSCs in spinner flask bioreactor, stirred bioreactor, or orbital shaker; and
   (iv) collecting the EVs from culture medium of the iPSCs.
23. The method of embodiment 22, wherein the iPSCs are derived from adult somatic cells.
24. The method of embodiment 23, wherein the adult somatic cells are fibroblasts or peripheral blood mononuclear cells.
25. The method of embodiment 22, wherein the iPSCs are cultured as 3D spheroids.
26. The method of embodiment 22, wherein the spinner flask bioreactor, stirred bioreactor, or orbital shaker is spun or stirred at a speed from about 30 rpm to about 300 rpm.
27. A method of treating an autoimmune disease or a disease characterized by increased inflammatory cytokine production, the method comprising administering to a subject in need thereof extracellular vesicles (EVs) derived from induced pluripotent stem cells (iPSCs).
28. The method of embodiment 27, wherein the iPSCs are derived from adult somatic cells.
29. The method of embodiment 28. wherein the adult somatic cells are fibroblasts or peripheral blood mononuclear cells.
30. The method of embodiment 27, wherein the iPSCs are cultured in spinner flask bioreactor, stirred bioreactor or orbital shaker.
31. The method of embodiment 27, wherein the EVs promote secretion of anti-inflammatory cytokines by macrophages.
32. The method of embodiment 31, wherein the anti-inflammatory cytokines comprise IL-10.
33. The method of embodiment 27, wherein the EVs regulate the activation and differentiation of T cells.
34. The method of embodiment 33, wherein the EVs promote differentiation of T cells into regulatory T cells.
35. The method of embodiment 33, wherein the EVs suppress secretion of inflammatory cytokines by T cells.
36. The method of embodiment 35, wherein the inflammatory cytokines comprise IFN-γ.
37. The method of embodiment 33, wherein the EVs promote secretion of anti-inflammatory cytokines by T cells.
38. The method of embodiment 37, wherein the anti-inflammatory cytokines comprise IL-10.
39. The method of embodiment 27, wherein the EVs are provided in a hydrogel composition.
40. The method of embodiment 39, wherein the hydrogel composition comprises laponite.
41. The method of embodiment 39, wherein the hydrogel composition further comprises chitosan.
42. The method of embodiment 27, wherein the autoimmune disease is type I diabetes, rheumatoid arthritis, psoriasis, Crohn's disease, lupus, or multiple sclerosis.
43. The method of embodiment 27, wherein the disease characterized by increased inflammatory cytokine production is septic shock, cytokine storm, or graft-versus-host disease.
44. A composition comprising extracellular vesicles (EVs) in a hydrogel matrix comprising laponite and chitosan, wherein the EVs are derived from induced pluripotent stem cells (iPSCs).
45. The composition of embodiment 44, wherein the iPSCs are derived from adult somatic cells.
46. The composition of embodiment 45, wherein the adult somatic cells are fibroblasts or peripheral blood mononuclear cells.
47. The composition of embodiment 44, wherein the iPSCs are cultured in spinner flask bioreactor, stirred bioreactor or orbital shaker.
48. The composition of embodiment 44, wherein the iPSCs are cultured as 3D spheroids.
49. The composition of embodiment 47, wherein the spinner flask bioreactor or stirred bioreactor or orbital shaker is spun or stirred at a speed from about 30-300 rpm.
50. The composition of embodiment 44 wherein the laponite concentration is 1-5 wt %.
51. The composition of embodiment 44 wherein the chitosan concentration is 0.5-5 wt %.

Various embodiments and aspects of the present invention as delineated hereinabove and as claimed in the claims section below find experimental support in the following examples.

### Example 1

Optimization of EV Isolation Method And Characterization of Isolated EVs

EVs derived from mesenchymal stem cells (MSCs) have been extensively studied for their immunoregulatory properties. For example, it was shown that MSC EVs can delay the onset of type 1 diabetes in mice by inducing the secretion of anti-inflammatory cytokines IL-10 and TGF-β, and reducing the production of pro-inflammatory cytokines (IFN-γ, or TNF-α). Moreover, MSC EVs have been reported to express PD-L1 and to induce tolerogenic signaling by promoting the generation of regulatory T cells (Tregs) and apoptosis of activated T cells. In contrast to MSC EVs, there are just few reports on EVs derived from iPSC. iPSC EVs have been shown to possess the potential to alleviate aging phenotype of senescent cells and to improve cardiac regeneration. However, their effect on immune cells remains largely unknown. One objective of the present example is, therefore, to compare the properties of MSC EVs and iPSC EVs. The concentration, size of EVs, their specific markers, and differences in the EV content between MSC EVs and iPSC EVs were evaluated.

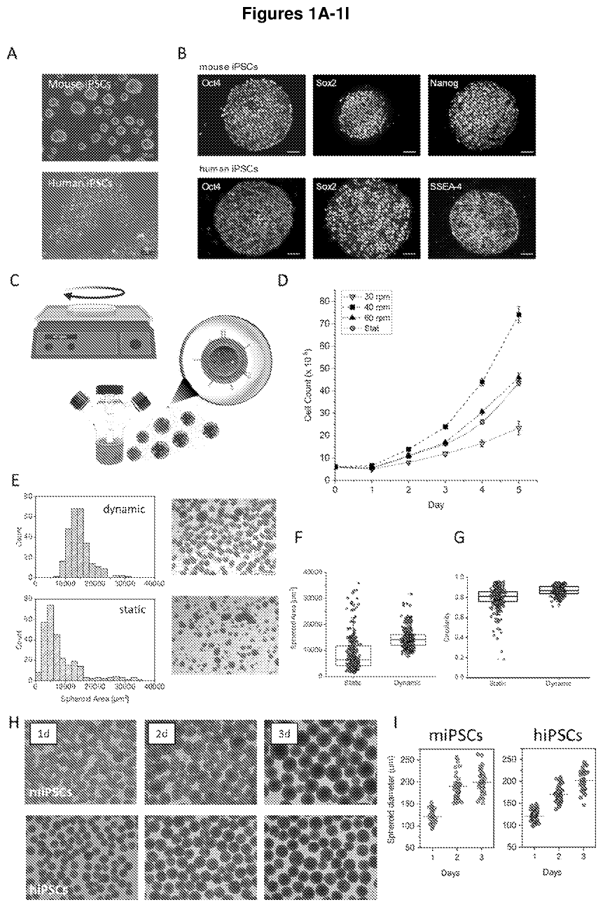

iPSCs were cultured under feeder-free conditions in a serum-free medium to eliminate the contamination of EVs from serum (FBS) (FIGS. 1A-I). For MSC cultures, medium supplemented with EV-depleted FBS was used, which was obtained by FBS ultracentrifugation at 100,000×g for 18 hrs at 4° C.

### Static vs Dynamic Cell Culture for EV Production

The therapeutic applicability of EVs is largely limited by the very low yield under standard culture conditions. Many cells and large volumes of culture media are needed in order to obtain a sufficient amount of EVs. In order to increase the production of EVs, the present example utilizes a method of cultivating cells in a spinner flask bioreactor or on an orbital shaker (dynamic culture). It was previously shown that a hollow fiber bioreactor can be used to increase the yield of EVs produced by HEK 293 cells, however, that type of bioreactor is not suitable for culture of naïve iPSCs as they grow in compact colonies. Therefore, a spinner flask bioreactor or an orbital shaker that enables 3D dynamic spheroid culture as well as 2D culture of cells on microcarrier beads was utilized for EV production (FIG. 1C). This type of bioreactor also permits easy sampling and cell viability assessment that is necessary for obtaining pure EVs without contaminations.

Microcarrier beads provide a support for the growth of iPSC cells as described here, for production of EVs. By way of on-limiting example, SoloHill plastic microcarriers beads (P-221-080, Sartorius AG) for iPSC dynamic culture for EV production were coated with 0.5 μg vitronectin (VTN-N; ThermoFisher Scientific) per cm² of microcarrier area. For the purposes disclosed herein, microcarrier beads may be sourced from any number of suppliers and may be fabricated from any number of plastics or polymers such as dextran. In some embodiments, the beads are coated with e.g., laminin, vitronectin, or collagen. Microcarrier beads are typically supplied having a diameter range, where typically at least 50% of the beads, or at least 70%, are of the labeled range. In one embodiments, the microcarrier beads are from about 80 to about 400 micrometers in diameter. In some embodiments the microcarrier beads are about 60 to about 87 micrometers in diameter. In some embodiments, the beads are about 125 to about 212 micrometers in diameter. In some embodiments the microcarrier beads are about 175 micrometers in diameter. In some embodiments the microcarrier beads are about 200 micrometers in diameter. Shakers, rollers or spinners, or stirred tank bioreactors may be used to increase the production of EVs by iPSCs in culture. Stirrers may also be employed. In one embodiment, a spinner for flasks is used. In one embodiment, orbital shaker may be used. In one embodiment a stirrer is used. In one embodiment the speed is between about 20 rpm and about 500 rpm. In one embodiment, the speed is about 30 rpm. In one embodiment, the speed is about 40 rpm. In one embodiment, the speed is about 50 rpm. In one embodiment, the speed is about 60 rpm. In one embodiment, the speed is about 100 rpm. In one embodiment, the speed is about 200 rpm. In one embodiment, the speed is about 300 rpm. In one embodiment, the speed is about 400 rpm. In one embodiment, the speed is about 500 rpm.

For administration and/or delivery of EVs as disclosed herein, any number of methods may be employed. EVs may be administered parenterally, e.g., by intravenous infusion. In some embodiments an EV delivery system comprises a controlled release hydrogel matrix. In one embodiment, EVs are encapsulated in a hydrogel, such as but not limited to a hydrogel comprising alginate, chitosan, laponite, hyaluronic acid, or any combination thereof. In one embodiment, EVs are encapsulated in a hydrogel, such as but not limited to a hydrogel comprising hydrophobically modified alginate, chitosan, hyaluronic acid, or any combination thereof. In some embodiments the hydrogel comprises laponite. In some embodiments, the hydrogel may be cross-linked by including a cross-linking agent such as Pluronic (e.g., Pluronic F127). Pluronics are non-ionic triblock copolymers composed of two hydrophilic side chains of poly (ethylene oxide), PEO, and a central hydrophobic chain of poly (propylene oxide), PPO (e.g., PEOx-PPOy-PEOx). They are also known as poloxamers and are non-toxic and biodegradable. By tuning the EV release properties of the hydrogel using polymers and cross-linkers, the desired features of administrability and duration of release can be provided. In some embodiments, about 5-25% Pluronic (e.g., F127) is included in the hydrogel. In some embodiments, about 15% Pluronic F127 is included.

In some embodiments, the hydrogel comprises hyaluronic acid. In some embodiments, the hydrogel comprises up to about 85% hyaluronic acid. In some embodiments, the hydrogel comprises about 5%, about 10%, about 15%, about 20%, about 25%, about 30%, about 35%, about 40%, about 45%, about 50%, about 55%, about 60%, about 65%, about 70%, about 75%, about 80, or about 85% hyaluronic acid. Percents here and elsewhere are wt % unless otherwise indicated.

In some embodiments, the hydrogel comprises a di-block copolymer or a tri-block copolymer. In some embodiments, the hydrogel comprises a non-ionic di-block copolymer or a non-ionic tri-block copolymer. In some embodiments, a non-ionic triblock copolymer such as Pluronic F147 is used. In some embodiments, the hydrogel comprises a non-ionic triblock copolymer such as Pluronic F147 up to about 30%. In some embodiments, the hydrogel comprises about 5%, about 10%, about 15%, about 20%, about 25%, about 30% non-ionic triblock copolymer. In some embodiments, the hydrogel comprises about 5%, about 10%, about 15%, about 20%, about 25%, about 30% Pluronic F147.

In some embodiments, the hydrogel comprise chitosan. In some embodiments the hydrogel comprises about 0.5 to about 5% chitosan. In some embodiments, the hydrogel comprises about 0.5%, about 0.6%, about 0.7%, about 0.8%, about 0.9%, about 1%, about 1.5%, about 2%, about 2.5%, about 3%, about 3.5%, about 4%, about 4.5%, or about 5% chitosan.

In some embodiments, the hydrogel comprises laponite. In some embodiments, laponite induces gelation. In some embodiments the hydrogel comprises about 1% to about 5% laponite. In some embodiments, the hydrogel comprises about 1%, about 1.5%, about 2%, about 2.5%, about 3%, about 3.5%, about 4%, about 4.5%, or about 5% laponite.

In some embodiments, the hydrogel comprise alginate. In some embodiments the hydrogel comprises about 0.5 to about 5% alginate. In some embodiments, the hydrogel comprises about 0.5%, about 0.6%, about 0.7%, about 0.8%, about 0.9%, about 1%, about 1.5%, about 2%, about 2.5%, about 3%, about 3.5%, about 4%, about 4.5%, or about 5% alginate. In some embodiments the alginate is crosslinked with a calcium salt. In some embodiments the calcium salt is calcium sulfate or calcium chloride. In some embodiments the calcium salt is provided at a concentration of about 10 to about 200 mM. In some embodiments, the calcium salt is provided at about 10 mM, about 20 mM, about 30 mM, about 40 mM, about 50 mM, about 60 mM, about 70 mM, about 80 mM, about 90 mM, about 100 mM, about 110 mM, about 120 mM, about 130 mM, about 140 mM, about 150 mM, about 160 mM, about 170 mM, about 180 mM, about 190 mM, or about 200 mM.

In some embodiments, the hydrogel comprises up to about 85% hyaluronic acid and about 5-30% non-ionic triblock copolymer such as Pluronic F147.

In some embodiments, the loading of the hydrogel with EV is about 5-250 micrograms of EVs per mL of hydrogel. In some embodiments, the loading of the hydrogel with EV is about 25-2500 micrograms of EVs per mL of hydrogel. In some embodiments, the loading is about 400 micrograms of EVs per mL of hydrogel. In some embodiments, the loading is about 500 micrograms of EVs per mL of hydrogel. In some embodiments the loading is about 1 mg of EVs per mL of hydrogel.

In some embodiments, the hydrogel comprises hyaluronic acid and Pluronic. In some embodiments, the hydrogel comprises about 85% hyaluronic acid and about 15% Pluronic F127.

In some embodiments, the hydrogel comprises chitosan and laponite.

In some embodiments, cross-linking of the hydrogel provides controlled release properties. In some embodiments, the hydrogel releases EVs over 1-7 days. In some embodiments the hydrogel releases EVs over 1-2 weeks. In some embodiments, at least two compositions are administered, one providing an immediate release of EVs and the other providing controlled release. The hydrogel is provided, in some embodiments, for administration parenterally such as but not limited to subcutaneously, intradermally, intravenously, intramuscularly, topically, nasally, intraperitoneally, or implanted in any organ or tissue.

Non-limiting examples of formulations of hydrogels for administering and delivering EVs include (values in wt % unless otherwise indicated):
0-85% hyaluronic acid, 5-30% Pluronic F147; example: 85% hyaluronic acid, 15% Pluronic F147;
0.5-5% chitosan, 1-5% laponite; example: 1% chitosan, 2% laponite;
0.5-5% alginate (10-200 mM CaSO4); example: 2% alginate (25 mM CaSO4).

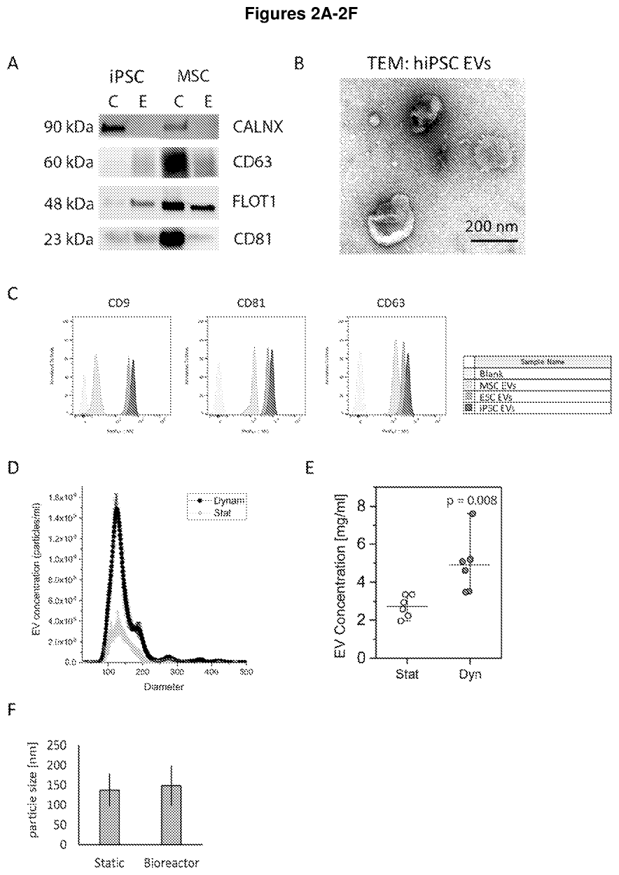

Mouse iPSC cells were cultured in the serum-free 2i medium and human iPSCs were cultured in mTeSR Plus or E8 media that maintains cells in the undifferentiated state (FIG. 1A), which was confirmed by fluorescence staining of pluripotency markers Oct4 and Sox2 (FIG. 1B). To increase the production of iPSC EVs, cells were cultured in spinner flask bioreactor or in suspension on an orbital shaker (FIG. 1C) at different agitation rates (dynamic culture). Forty rpm was determined as the optimal agitation rate that significantly increases the iPSC growth (FIG. 1D) and permits cell aggregation into spheroids with homogenous size distribution (FIG. 1E). In contrast to static iPSC culture, dynamic stimulation enables formation of spheroids with increased area and more homogenous size distribution (FIG. 1F) and better circularity (FIG. 1G), which facilitates uniform cell growth. Small EVs of a size less than 200 nm were isolated from the conditioned media by differential ultracentrifugation, gradient ultracentrifugation or by ultrafiltration and size exclusion chromatography. To demonstrate the presence of EVs, EV markers such as CD9, CD63, CD81 or Flot 1 were evaluated by western blot and flow cytometry (FIG. 2A, 2C; peaks are, left to right, Blank, MSC EVs and iPSC EVs.). The vesicular nature of isolated particles was confirmed by transmission electron microscopy (FIG. 2B). The concentration of EVs was measured by nanoparticle tracking analysis (NTA) or by resistive pulse sensing (RPS) or by microBCA (FIG. 2D, 2E). It was confirmed that iPSCs cultured under dynamic conditions secreted approx. two times more EVs as cells grown under static conditions after 24 h. The mean size of these EVs was around 150 nm and it didn't differ between static and dynamic culture conditions (FIG. 2F).

A comprehensive integrated analysis of the vesicular transcriptome and proteome can be used to unveil the components that contribute to the immunomodulatory properties of EVs. For example, next generation sequencing can be used to profile expression of regulatory RNAs, and bioinformatics tools such as TargetScan or ComiR can be used to predict target genes of miRNAs enriched in EVs. miRNAs as important regulators of gene expression can be packed into EVs and influence immune cell differentiation, function, and homeostasis.

To gain further knowledge about the protein content of both MSC and iPSC EVs, liquid chromatography-tandem mass spectrometry (LC-MS/MS) can be used to assess the proteomic profile of EVs. Proteins can be classified based on their function and cellular localization using Protein Analysis Through Evolutionary Relationships (PANTHER). Immune system-related proteins can be assessed using Immuno-Navigator database. Protein-protein interaction (PPI) network of candidate proteins can be generated using String database web tool. To identify molecular pathways involved in the immunoregulatory functions of EVs, proteins can be categorized using Kyoto Encyclopedia of Genes and Genomes (KEGG) Mapper tool.

Characterization of iPSC EVs

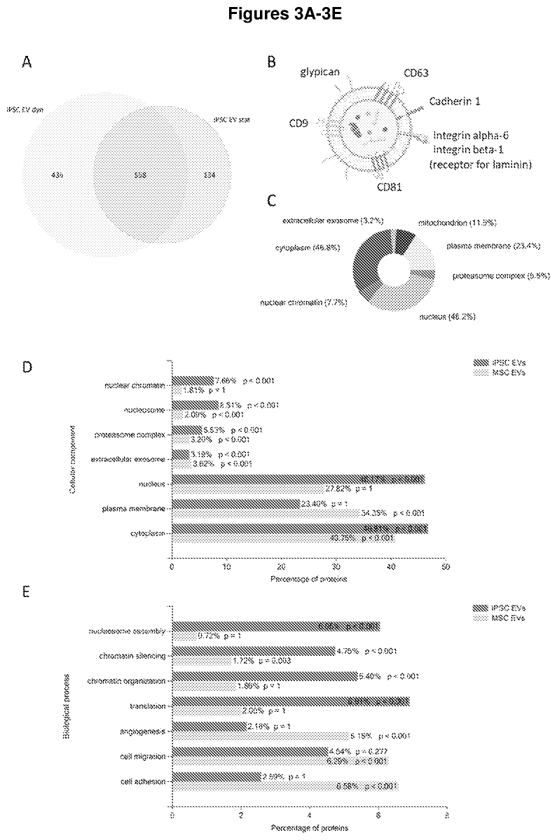

The content of iPSC EVs from static and dynamic cultures was analyzed by mass spectrometry (MS). iPSC EVs from dynamic culture contained majority of the proteins detected in static iPSC EVs and additional proteins that were not detected in static iPSCs (FIG. 3A). Among the iPSC EV surface proteins are, for example, the well-known EV markers such as, CD9, CD63 and CD81, but also proteins involved in cell adhesion such as cadherin 1 or integrin alpha 6/beta 1 (FIG. 3B). iPSC EVs contain proteins derived from cell nucleus, nuclear chromatin, cytoplasm, mitochondrion, plasma membrane, and also proteasome components (FIG. 3C). In contrast to MSC EVs, iPSC EVs contain significantly more nuclear proteins (FIG. 3D), which are involved in nucleosome assembly, chromatin organization and gene silencing. On the other hand, in comparison to MSC EVs, iPSC EVs contain less cell adhesion proteins or proteins involved in angiogenesis (FIG. 3E).

### Example 2

### Encapsulation And Release of iPSC EVs

In the present example, hydrogels were utilized as carriers for EV delivery. It has been shown that sustained presentation of cytokines can alter the biological behaviors of immune cells compared to those provided in soluble forms. Various 2D or 3D biomaterials have been used as localized delivery platforms for natural nanostructures like viruses. However, biomaterials have not been used for localized delivery of iPSC EVs for immunomodulation so far. Delivering EVs through biomaterials can provide protection for the EVs and enable better control of local EV concentration by preventing burst dilution (leakage) in body fluids or blood.

In one embodiment, in situ formed hydrogel based on chitosan and laponite (FIG. 4) can be used. Chitosan as a biocompatible biopolymer has been frequently used to synthesize physically or chemically cross-linked hydrogels for various biomedical applications. Chitosan's cationic nature can provide binding sites to interact and physically immobilize encapsulated EVs. In order to prepare the in situ forming gel, chitosan can be chemically modified with hydrophobic chains as known in the art. However, this self-assembly provides a very compact meso-/nanoscopic mesh structure (ca. 3 nm between the nanodomains) which might inhibit the release of nanoscale objects like EVs. To overcome this issue, laponite can be used. Laponite is a synthetic nanosilicate with a disc-shaped morphology and dual charged surface, which facilitates gelation at low concentrations (e.g., 2 wt %) in aqueous environment. Since the laponite are synthetic polyions, they act as multifunctional cross-linkers to the chitosan chains through hydrogen bonding and ionic interactions. The cross-linking is reversible because under deformation, the chitosan chains may attach and detach from the nanoparticle surfaces. Crosslinked HA can control release and bioavailability of encapsulated reagents, e.g., EVs. However, the chemical crosslinking can affect the bioactivity of encapsulated protein-containing therapeutics. Therefore, physically crosslinked hydrogels can be utilized as EV delivery platforms. Such hydrogel can be prepared by the addition of another polymer called Pluronic to the HA formulation. Pluronics (PEOx-PPOy-PEOx) are non-ionic triblock copolymers composed of two hydrophilic side chains of poly (ethylene oxide), PEO, and a central hydrophobic chain of poly (propylene oxide), PPO. They are also known as poloxamers and are non-toxic and biodegradable. One of the main characteristics of Pluronic F127 is that it forms a gel at relatively low concentrations and temperatures close to room temperature, which is very interesting for biomedical applications. However, Pluronic hydrogel structure is not maintained for a long time in physiological conditions, due to the low molecular weight and low mechanical strength, which result in burst drug release. Incorporation of HA can fix this issue as incorporated HA chains can bridge Pluronic micelles and stabilize the hydrogel to provide 1-2 weeks of sustained release. We have demonstrated the feasibility of this approach (FIG. 4D).

In one embodiment, the EVs can be encapsulated in hydrogels to provide sustained release. For example, 3% (wt/vol) suspensions of Laponite XLG (obtained from Southern Clay Products, Inc. Louisville) were prepared by vigorous vortexing of Laponite powder in deionized water. Suspensions were autoclaved and volume adjusted with sterile deionized water. Control hydrogels were prepared based on 1.5 (wt/vol) GMP-grade alginate (PRONOVA UP MVG, NovaMatrix, Norway) in sterile deionized water. Hydrogels were crosslinked using 25 mM of CaSO4. To prepare EV encapsulated hydrogels, iPSC EVs were premixed with either of laponite or alginate at concentration of 50 μg/ml for in vitro release studies and 1 mg/ml for in vivo experiments.

To assess the release of iPSC EVs from hydrogels, EVs was premixed with by Laponite or alginate at a concentration of 40 μg/ml. One hundred μl aliquots of hydrogel-EVs were casted into low-protein binding 96 well-plates and transferred into 50 ml Falcon tube after gelation which contains 10 ml of PBS. The tubes were kept at 37° C. incubators under gentle shaking (100 rpm) and the media was recovered at various time points and stored at −20° C. until analysis using micro-BCA assay. The results were shown in FIG. 4B.

### Example 3

### Immunomodulatory Properties of iPSC EVs

In the present example, immunomodulatory properties of iPSC EVs were evaluated by co-incubation of iPSC EVs with macrophages and T cells.

Macrophages are generally reported to be among the first cells to internalize EVs when administered in the body. They can be the initiators of inflammatory responses, but they have also important role in the maintenance of tissue homeostasis and regeneration. Two major macrophage populations have been defined: the classically activated macrophages that respond to intracellular pathogens by secreting proinflammatory cytokines (also known as M1 macrophages), and alternatively activated macrophages which display anti-inflammatory activities (also known as M2 macrophages).

Immunomodulatory properties of free EVs as well as those loaded and delivered via hydrogel platform were examined. Costar transwell inserts (pore size 1 μm) were used to evaluate the effect of EVs on polarization of macrophages and differentiation of T cells. Free and hydrogel-encapsulated EVs at different concentrations (1-200 μg/ml) can be placed in the upper chamber. The bottom chamber can contain 10⁵ of either primary monocytes or naïve T cells or both (5×10⁴ each). The isolation of primary monocytes can be performed as generally known in the art. Naïve T cells can be isolated using EasySep mouse or human T Cell Isolation Kit (STEMCELL Technologies) according to manufacturer's protocol. The change in macrophage phenotype was tested at different time points (day 1-5) using flow cytometry by checking representative surface markers: major histocompatibility complex class II (MHC II), CCR7, CD80 and CD86 for M1 macrophages; CD163 (M2c) and CD206 (M2a) for M2 macrophages. The change in macrophage function can be characterized by phagocytosis capability, inducible nitric oxide synthase (iNOS) production and inflammatory secretome analysis (IL-1-0, TNF, IL-6, and IL-12 for M1 macrophages; IL-4, IL-10, and TGF-β for M2 macrophages). Quantitative PCR (qPCR) can also be used to determine the gene expression of these inflammatory cytokines and cellular markers. Activation and differentiation of T cells can be tested by considering expression of CD25 and CD44 surface activation marker as well as intracellular expression of the transcription factor Foxp3. In addition, soluble EVs can be used as controls to compare with the bioactivity of encapsulated EVs.

### Macrophage Activation and Differentiation

Human monocytic THP-1 cells were maintained in culture in RPMI 1640 culture medium containing 10% FBS supplemented with 10 mM HEPES, 1 mM pyruvate, 2.5 g/l D-glucose and 50 pM ß-mercaptoethanol. THP-1 monocytes were differentiated into macrophages by 24 hrs incubation with 50 nM PMA followed by 48-72 hrs incubation (resting) in RPMI medium. Macrophages were polarized to M1 macrophages by incubation with 20 ng/ml of IFN-γ and 10 μg/ml of LPS for 24 h. Macrophage M2 polarization was obtained by incubation with 20 ng/ml of IL-4 and 20 ng/ml of IL-13 for 24 h. Cell density of 1×10⁶ in 24 well plate was used in all monocyte/macrophage experiments. In the co-culture experiments, THP-1 monocytes were co-incubated with iPSC or MSC EVs for 24 hrs. After incubation with or without EVs, the cells were washed with cold PBS and detached with EDTA (5 mM) and stained for flow cytometry. Flow cytometry was performed on a Cytek DXP 10. FACS data were analyzed using FlowJo software (Treestar).

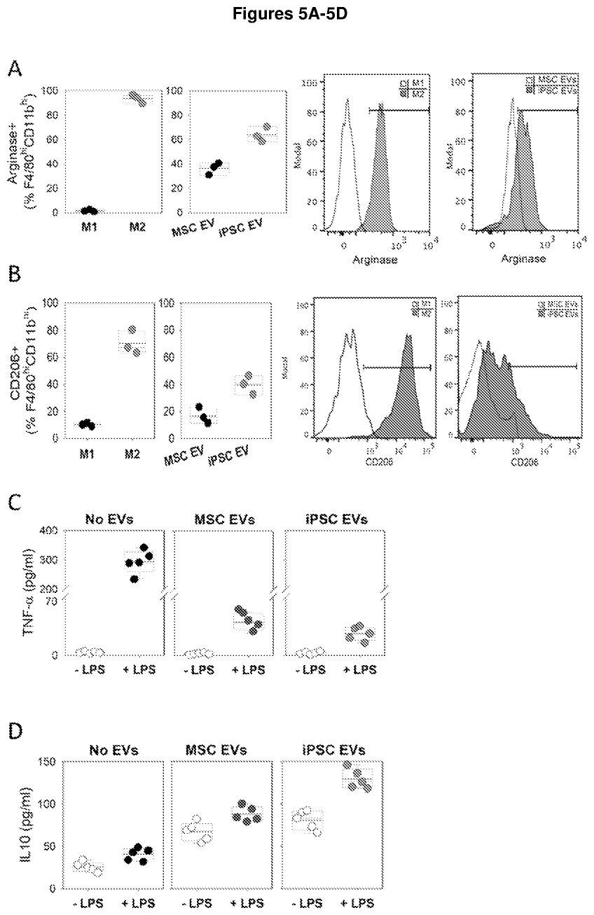

As shown in FIG. 5, iPSC EVs can promote differentiation of macrophage toward anti-inflammatory/pro-healing macrophages (M2). Upregulation of Arginase (FIG. 5A) and CD206 (FIG. 5B) surface markers were shown. These two markers are widely used as M2 macrophages markers.

Cytokine secretion in the culture medium was assayed using an ELISA kit. As shown in FIGS. 5C-D, iPSC EVs not only suppress secretion of inflammatory cytokines (TNF-alpha) but also induce production of anti-inflammatory cytokines (IL-10) by macrophages in vitro.

### T Cell Isolation and Activation

Five to eight-week-old wild-type (C57Bl/6) mice were purchased from the Jackson Labs and maintained in specific pathogen-free facilities at UCLA. All experiments on mice and cells collected from mice were performed under an approved protocol of the Animal Research Committee and in accordance with UCLA's institutional policy on humane and ethical treatment of animals. T cell culture media was RPMI supplemented with 10% heat-inactivated FBS, 1% penicillin/streptomycin, 1% sodium pyruvate, 1% HEPES buffer, 0.1% μM beta-mercaptoethanol. Total T cells, CD4⁺ T cells were purified using magnetic-based, negative enrichment kits (Stem Cell Technologies). Cells were counted by hemocytometer using trypan blue exclusion (Calbiochem).

In vitro activation of purified T cells was done by culturing cells at a concentration of 1.5×10⁶/mL in 24-well plate coated with anti-CD3 (2C11; Bio X Cell) at a concentration of 10 μg/mL followed by addition of 2 μg/mL soluble anti-CD28 (37.51; Bio X Cell). iPSC EVs were added to the T cell media during the cell seeding. Twenty IU/mL of human IL-2 was also added. For flow cytometric analyses, antibodies to mouse CD4 (53-6.7), CD25 (PC61.5), CD44 (IM7), FoxP3 and CD16/CD32 (“Fc block”) were purchased from BioLegend.

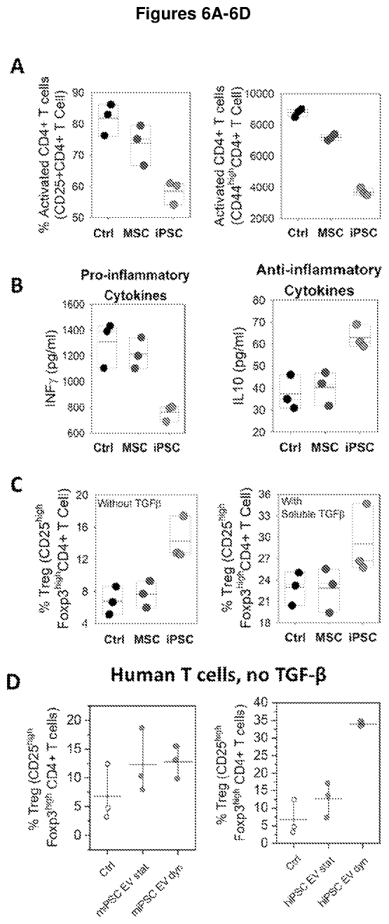

Cells were analyzed by flow cytometry after 3 days of culture. Flow cytometry was performed on a Cytek DXP 10. FACS data were analyzed using FlowJo software (Treestar). As shown in FIG. 6A, iPSC EVs can suppress activation of CD4 T cells.

Secretion of inflammatory (IFN-γ) and anti-inflammatory (IL-10) cytokines was tested using ELISA kits. The cytokines were measured after priming primary CD4⁺ T cells with anti-CD3 and anti-CD28 in the presence of 20 U/mL IL-2. As shown in FIG. 6B, iPSC EVs not only suppress secretion of inflammatory cytokines (IFN-γ) but also induce production of anti-inflammatory cytokines (IL-10) by T cells in vitro.

To induce regulatory T cells (iTreg), CD4⁺ T cells were purified from mouse spleens or from human blood by EasySep immunomagnetic negative selection (Stem Cell Technologies). The cells were then activated on anti-CD3 antibody (10 μg/mL)-coated plates with media supplemented with anti-CD28 antibody (2 μg/mL) as mentioned above. iPSC or MSC EVs and IL-2 (20 IU/mL) were added to the media with or without TGF-β. After four days, regulatory T cells were removed from wells and fixed, permeabilized, and stained with CD25 surface and Foxp3 intracellular antibodies for flow cytometry analysis. As shown in FIG. 6C, iPSC EVs can promote formation of regulatory T cells (Tregs) in the presence or absence of TGF-β.

FIG. 6D shows differences between the effects of mouse and human iPSC EVs isolated from static and dynamic cultures on formation of human regulatory T cells in the absence of TGF-β. Mouse iPSC EVs isolated from static (miPSC EV stat) and dynamic (miPSC EV dyn) cultures can increase the formation of human Tregs to the same extent as human iPSC EVs isolated from static culture (hiPSC EV stat) while human iPSC EVs isolated from dynamic culture (iPSC EV dyn) are more powerful in promoting formation of human regulatory T cells (FIG. 6D).

### Immunomodulatory Properties In Vivo

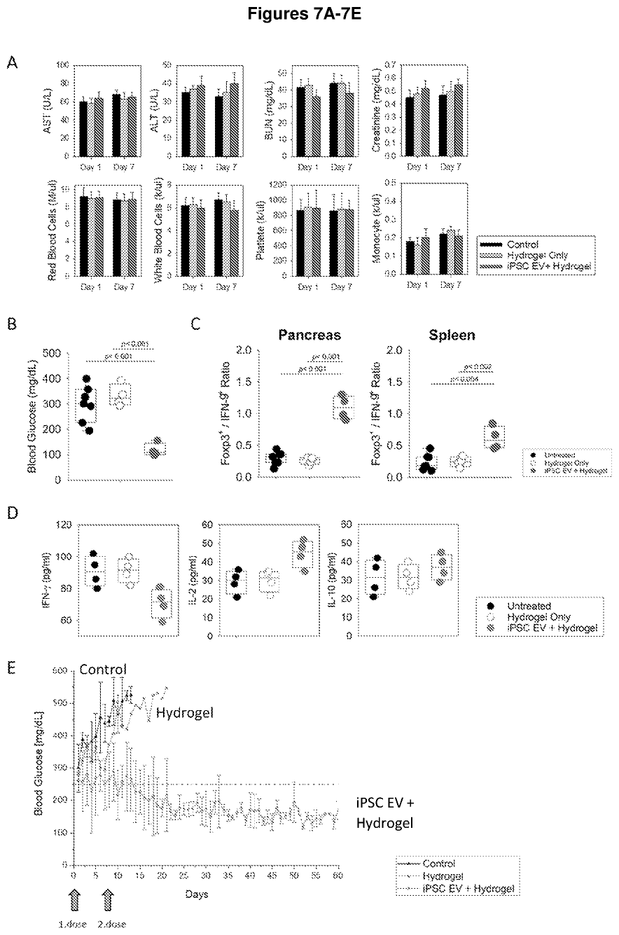

Evaluation of safety and toxicity of EV-based therapy was performed in C57BL/6 mice. Toxicology profile was evaluated using whole blood test. Blood samples were drawn from the retro-orbital sinus at different times (day 1 and day 7), collected in EDTA tubes, maintained for 30 min and centrifuged at 10,000 g for 10 min. The toxicity profile was measured using Drew Scientific HemaVet 950FS blood analyser. As shown in FIG. 7A, no systematic toxicity was detected after administration of iPSC EVs. Each set of three bars shows, left to right, Control, Hydrogel only and iPSC EV+hydrogel.

Evaluation of immunomodulatory properties of iPSC EVs in vivo was examined in two mouse models of T1D (type 1 diabetes): T cell transfer model (adaptive transfer mouse model) and a non-obese diabetic (NOD) mouse model, i.e., a natural model with certain parallels to the human disease including the generation of autoreactive T and B cells specific for islet autoantigens. Female NOD/scid mice (8 weeks old) and female NOD/LtJ mice (15 weeks old) from Jackson Lab were used for an adoptive transfer model. To induce T1D, 5×10⁶ splenocytes from pre-diabetic NOD/LtJ mice were intravenously injected into NOD/scid mice. Two weeks after adoptive transfer, the control group were left untreated, and the other groups were subcutaneously injected with 100 μL of hydrogels alone or 100 μg hydrogel-encapsulated iPSC EVs. Hydrogel formulations can be injected at the subcutaneous dorsal neck region. Subsequently, the glucose levels of the mice were monitored every two days to the endpoint. Mice treated with human iPSC EVs+hydrogel kept their blood glucose level normal until the end of the experiment while untreated mice and mice with hydrogel alone were hyperglycemic (FIG. 7B).

The spleens and pancreases of the mice were collected and analyzed for the status of infiltrating T cells. The spleens and pancreas were dissociated to obtain single cells followed by incubating in ACK lysis buffer (Gibco) for 5 min at room temperature to remove red blood cells. Subsequently, the cells were stained with CD3, CD4, and CD25 antibodies. For intracellular cytokine staining (ICCS), cells were fixed with cold 4% paraformaldehyde (PFA) for 20 min at room temperature and then permeabilized with 0.5% saponin for 10 min before blocking with PBS+1% BSA+5% donkey serum and staining with appropriate antibodies at 1:100 dilution. The cells were washed and analyzed by flow cytometry. The following antibodies from Biolegend were used for ICCS: anti-IFN-γ and anti-Foxp3. The percentages of IFN-γ+CD4⁺ T cells (defined as effector T cells) and FoxP3⁺CD25⁺CD4⁺ T cells (defined as regulatory T cells (Treg)) were determined (FIG. 7C). Secretion of inflammatory (IFN-gamma) and anti-inflammatory (IL-2 and IL-10) cytokines was tested using ELISA kit after stimulating extracted cells from pancreases with LPS overnight (FIG. 7D).

As shown in FIG. 7D, iPSC EVs can suppress secretion of inflammatory cytokines (IFN-γ) but promote production of anti-inflammatory cytokines (IL-2) by immune cells. In FIGS. 7B, C and D, each graph shows data for, left to right, Untreated, Hydrogel Only and iPSC EV+Hydrogel.

As a second model of T1D, NOD mice that spontaneously develop T1D were used with similar experimental groups as the case of adaptive transfer model. Blood glucose levels of NOD mice will be monitored from 10 weeks of age. Once the mice were hyperglycemic (>250 mg/dL) for two days, the control group was left untreated while the other groups were injected with hydrogel alone or hydrogel-encapsulated iPSC EVs and the second dose was administered 7 days after the first dose. As shown on FIG. 7E, the blood glucose level of NOD mice treated with iPSC EVs+hydrogel gradually returned back to normal and the mice remained normoglycemic for 60 days while the blood glucose level of untreated NOD mice and mice treated with hydrogel alone gradually increased and they needed to be sacrificed before the end of the experiment.

### Example 5

### EVs Derived from Embryonic Stem Cells

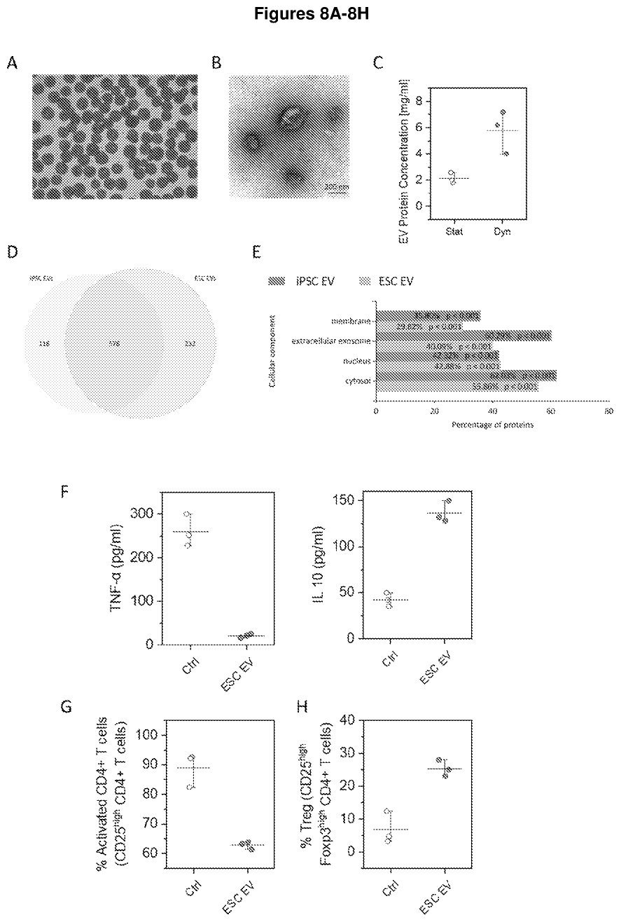

In the present example, ESCs were cultured under feeder-free conditions in a serum-free medium such as E8 medium or mTeSR Plus medium (FIGS. 8A-8H). The dynamic culture and EV yields from ESCs were similar to iPSC cells and also the composition of iPSC and ESC EVs was very similar. In a spinner flask bioreactor, ESCs were grown on microcarrier beads or as 3D spheroids and EVs were isolated from medium conditioned by cells. By way of on-limiting example, SoloHill plastic microcarriers beads (P-221-080, Sartorius AG) for ESC dynamic culture for EV production were coated with 0.5 μg vitronectin (VTN-N; ThermoFisher Scientific) per cm² of microcarrier area. For the purposes disclosed herein, microcarrier beads may be sourced from any number of suppliers and may be fabricated from any number of plastics or polymers such as dextran. In some embodiments, the beads are coated with e.g., laminin, vitronectin, or collagen. Microcarrier beads are typically supplied having a diameter range, where typically at least 50% of the beads, or at least 70%, are of the labeled range. In one embodiments, the microcarrier beads are from about 80 to about 400 micrometers in diameter. In some embodiments the microcarrier beads are about 60 to about 87 micrometers in diameter. In some embodiments, the beads are about 125 to about 212 micrometers in diameter. In some embodiments the microcarrier beads are about 175 micrometers in diameter. In some embodiments the microcarrier beads are about 200 micrometers in diameter. Shakers, rollers or spinners, or stirred tank bioreactors may be used to increase the production of EVs by ESCs in culture. Stirrers may also be employed. In one embodiment, a spinner for flasks is used. In one embodiment, orbital shaker may be used. In one embodiment a stirrer is used. In one embodiment the speed is between about 30 rpm and about 500 rpm. In one embodiment, the speed is about 30 rpm. In one embodiment, the speed is about 40 rpm. In one embodiment, the speed is about 50 rpm. In one embodiment, the speed is about 60 rpm. In one embodiment, the speed is about 100 rpm. In one embodiment, the speed is about 200 rpm. In one embodiment, the speed is about 300 rpm. In one embodiment, the speed is about 400 rpm. In one embodiment, the speed is about 500 rpm.

Immunomodulatory properties of ESC EVs were evaluated by co-incubation of ESC EVs with macrophages and T cells. Similarly to iPSC EVs, ESC EVs were able to polarize macrophages into anti-inflammatory phenotype (M2) documented by increased secretion of anti-inflammatory cytokines such as IL-10 (FIG. 8F) and decreased production of proinflammatory cytokines such as TNF-α by macrophages activated with bacterial lipopolysaccharide (LPS).

In vitro activation of purified T cells was done by culturing cells at a concentration of 1.5×10⁶/mL in 24-well plate coated with anti-CD3 (2C11; Bio X Cell) at a concentration of 10 μg/mL followed by addition of 2 μg/mL soluble anti-CD28 (37.51; Bio X Cell). T cell culture medium was also supplemented with 20 IU/mL of human IL-2. ESC EVs (100 ug/ml) were added to the T cell media during the cell seeding. Cells were analyzed by flow cytometry after 3 days of culture. Flow cytometry was performed on a Cytek DXP 10. FACS data were analyzed using FlowJo software (Treestar). As shown in FIG. 8G, similarly to iPSC EVs, ESC EVs can suppress activation of CD4 T cells. ESC EVs are also able to induce the formation of regulatory T cells (Tregs) (FIG. 8H).

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

What is claimed is:

1. A method of treating an autoimmune disease or a disease characterized by increased inflammatory cytokine production, the method comprising administering to a subject in need thereof extracellular vesicles (EVs) derived from induced pluripotent stem cells (iPSCs) or embryonic stem cells (ESCs).

2. The method of claim 2, wherein the iPSCs are derived from fibroblasts, peripheral blood mononuclear cells, myoblasts, keratinocytes, melanocytes, hepatocytes, β-cells, dental pulp cells, blood cells, urine-derived renal epithelial cells, amniotic fluid stem cells, muscle cells, or adult stem cells.

3. The method of claim 2, wherein the iPSCs or ESCs are cultured in dynamic culture such as in a spinner flask bioreactor, stirred bioreactor or orbital shaker.

4. The method of claim 3, wherein the spinner flask bioreactor or stirred tank bioreactor or orbital shaker is spun or stirred at about 20-500 rpm.

5. The method of claim 1, wherein the iPSCs or ESCs are cultured on microcarrier beads or as 3D spheroids.

6. The method of claim 1, wherein the EVs promote secretion of anti-inflammatory cytokines by macrophages.

7. The method of claim 6, wherein the anti-inflammatory cytokines comprise IL-10.

8. The method of claim 1, wherein the EVs regulate the activation and differentiation of T cells.

9. The method of claim 8, wherein the EVs promote differentiation of T cells into regulatory T cells.

10. The method of claim 8, wherein the EVs suppress secretion of inflammatory cytokines by T cells.

11. The method of claim 10, wherein the inflammatory cytokines comprise IFN-gamma.

12. The method of claim 1, wherein the EVs promote secretion of anti-inflammatory cytokines by T cells.

13. The method of claim 12, wherein the anti-inflammatory cytokines comprise IL-10.

14. The method of claim 1, wherein the EVs are provided in a hydrogel composition.

15. The method of claim 14, wherein the hydrogel composition comprises hyaluronic acid, a di-block copolymer, a tri-block co-polymer, laponite, a self-assembly peptide, an optionally hydrophobically-modified biopolymers such as chitosan, hyaluronic acid, and alginate, or any combination thereof.

16. The method of claim 1, wherein the autoimmune disease is type I diabetes, rheumatoid arthritis, psoriasis, Crohn's disease, periodontitis, gingivitis, asthma, Alzheimer's disease, Parkinson's disease, systemic lupus erythematosus, multiple sclerosis, or protection of tissue graft after transplantation (for example, beta cells made by differentiation of iPSCs or embryonic stem cells).

17. The method of claim 1, wherein the disease characterized by increased inflammatory cytokine production is septic shock, cytokine storm, or Graft-Versus-Host Disease.

18. A method of producing extracellular vesicles (EVs) derived from induced pluripotent stem cells (iPSCs) or embryonic stem cells (ESCs), comprising the steps of
(i) culturing the iPSCs or ESCs by dynamic culture such as by spinner flask bioreactor, stirred tank bioreactor or orbital shaker; and
(ii) collecting the EVs from culture medium of the iPSCs or ESCs.

19. The method of claim 18, wherein the iPSCs are derived from fibroblasts, peripheral blood mononuclear cells, myoblasts, keratinocytes, melanocytes, hepatocytes, O-cells, dental pulp cells, blood cells, urine-derived renal epithelial cells, amniotic fluid stem cells, muscle cells, or adult stem cells.

20. The method of claim 18, wherein the iPSCs or ESCs are cultured on microcarrier beads or as 3D spheroids.

21. The method of claim 20, wherein the microcarrier beads are about 80 to about 400 μm in diameter.

22. The method of claim 18, wherein the spinner flask bioreactor or stirred bioreactor or orbital shaker is spun or stirred at about 20 to about 500 rpm.

23. A composition comprising extracellular vesicles (EVs) derived from induced pluripotent stem cells (iPSCs) or embryonic stem cells (ESCs).

24. The composition of claim 23, wherein the iPSCs are derived from fibroblasts, peripheral blood mononuclear cells, myoblasts, keratinocytes, melanocytes, hepatocytes, β-cells, dental pulp cells, blood cells, urine-derived renal epithelial cells, amniotic fluid stem cells, muscle cells, or adult stem cells.

25. The composition of claim 23, wherein the iPSCs or ESCs are cultured in spinner flask bioreactor, orbital shaker or stirred bioreactor.

26. The composition of claim 23, wherein the iPSCs or ESCs are cultured on microcarrier beads or as 3D spheroids.

27. The composition of claim 26, wherein the microcarrier beads are about 80 to about 400 μm in diameter.

28. The composition of claim 25, wherein the spinner flask bioreactor or stirred bioreactor or orbital shaker is spun or stirred at about 20-500 rpm.

29. The composition of claim 23, wherein the EVs regulate the activation and differentiation of macrophages.

30. The composition of claim 29, wherein the EVs promote differentiation of macrophages into M2 macrophages.

31. The composition of claim 29, wherein the EVs suppress secretion of inflammatory cytokines by macrophages.

32. The composition of claim 31, wherein the inflammatory cytokines comprise TNF-alpha.

33. The composition of claim 23, wherein the EVs promote secretion of anti-inflammatory cytokines by macrophages.

34. The composition of claim 33, wherein the anti-inflammatory cytokines comprise IL-10.

35. The composition of claim 23, wherein the EVs regulate the activation and differentiation of T cells.

36. The composition of claim 35, wherein the EVs promote differentiation of T cells into regulatory T cells.

37. The composition of claim 35, wherein the EVs suppress secretion of inflammatory cytokines by T cells.

38. The composition of claim 37, wherein the inflammatory cytokines comprise IFN-gamma.

39. The composition of claim 23, wherein the EVs promote secretion of anti-inflammatory cytokines by T cells.

40. The composition of claim 39, wherein the anti-inflammatory cytokines comprise IL-10.

41. The composition of claim 23, further comprising a hydrogel composition.

42. The composition of claim 41, wherein the hydrogel composition comprises laponite/hyaluronic acid, a self-assembly polymer such as a bi-block or tri-block polymer, a self-assembly peptides, an optionally hydrophobically-modified biopolymer such as chitosan, hyaluronic acid, and alginate, or any combination thereof.

43. A composition comprising extracellular vesicles (EVs) in a hydrogel matrix wherein the EVs are derived from induced pluripotent stem cells (iPSCs) or embryonic stem cells (ESCs).

44. The composition of claim 43, wherein the iPSCs are derived from fibroblasts, peripheral blood mononuclear cells, myoblasts, keratinocytes, melanocytes, hepatocytes, β-cells, dental pulp cells, blood cells, urine-derived renal epithelial cells, amniotic fluid stem cells, muscle cells, or adult stem cells.

45. The composition of claim 43, wherein the iPSCs or ESCs are cultured in dynamic culture such as in a spinner flask bioreactor or stirred tank bioreactor or orbital shaker.

46. The composition of claim 43, wherein the iPSCs or ESCs are cultured on microcarrier beads or as 3D spheroids.

47. The composition of claim 46, wherein the microcarrier beads are about 80 to about 400 μm in diameter.

48. The composition of claim 45, wherein the spinner flask bioreactor or stirred bioreactor or orbital shaker is spun or stirred at 20-500 rpm.

49. The composition of claim 43 wherein the hydrogel composition comprises hyaluronic acid, self-assembly polymer such as a di-block copolymer, a tri-block copolymer, laponite, a self-assembly peptide, an optionally hydrophobically-modified biopolymer such as chitosan, hyaluronic acid, and alginate, or any combination thereof.

50. The composition of claim 43 wherein the concentration of EVs in the composition is about 0.05 mg to about 2.5 mg per mL of hydrogel.

51. The composition of claim 43 comprising 0-85% hyaluronic acid and 5-30% Pluronic F147.

52. The composition of claim 51 comprising 85% hyaluronic acid and 15% Pluronic F147.

53. The composition of claim 45 comprising 0.5-5% chitosan and 1-5% laponite.

54. The composition of claim 53 comprising 1% chitosan and 2% laponite.

55. The composition of claim 45 comprising 0.5-5% alginate and 10-200 mM CaSO4.

56. The composition of claim 45 comprising 2% alginate and 25 mM CaSO4.
